SEQUENCE LISTING (6 sequences)

SEQ ID NO: 1 - DNA, 26 bp, Artificial Sequence
  Synthetic
agaaagttgtcagaccctagaccaaa

SEQ ID NO: 2 - DNA, 24 bp, Artificial Sequence
  Synthetic
ggtagggagggaaacattgtcaca

SEQ ID NO: 3 - DNA, 22 bp, Artificial Sequence
  Synthetic
aggacaggttgaggctggtaca

SEQ ID NO: 4 - DNA, 24 bp, Artificial Sequence
  Synthetic
ggtagagtgggttgaggttggaaa

SEQ ID NO: 5 - DNA, 22 bp, Artificial Sequence
  Synthetic
cagcaggctctcagtcctttcc

SEQ ID NO: 6 - DNA, 20 bp, Artificial Sequence
  Synthetic
tccagtcacaggtgccgtct

(19) United States
(12) Patent Application Publication
(10) Pub. No.: US 2010/0272824 A1
(43) Pub. Date: Oct. 28, 2010

(54) COMPOSITIONS AND METHODS FOR PREVENTING AND MONITORING DISEASE

(75) Inventors: Joanne R. Lupton, College Station, TX (US); Nancy D. Turner, College Station, TX (US); Youngmi Cho, College Station, TX (US); Hyemee Kim, College Station, TX (US)

Correspondence Address: MEDLEN & CARROLL, LLP, 101 HOWARD STREET, SUITE 350, SAN FRANCISCO, CA, 94105, US

(73) Assignee: The Texas A&M University System

(21) Appl. No.: 12/761,234

(22) Filed: Apr. 15, 2010

Related U.S. Application Data
(60) Provisional application No. 61/170,026, filed on Apr. 16, 2009.

(51) Int. Cl. A61K 35/60; C40B 30/00; A61P 35/00

(52) U.S. Cl. 424/523; 506/7

(57) ABSTRACT

Gene expression patterns were analyzed that identified multiple genes associated with radiation enhanced colon carcinogenesis. Some of the genes modulated by radiation exposure may be involved in up-regulating the Wnt/β-catenin signaling pathway and promote colon carcinogenesis. Consumption of a fish oil/pectin diet caused a general down-regulation of genes encoding proteins involved in cell adhesion and receptor activity, which may be involved in down-regulating the Wnt/β-catenin signaling pathway. The data suggest that dietary fish oil/pectin may be an effective countermeasure against radiation-induced colon carcinogenesis. In accordance with this, gene expression profiles can be monitored to reduce the risk of radiation induced carcinogenesis in high risk personnel (i.e., for example, astronauts) before, during, and/or after radiation exposure (i.e., for example, spaceflight), and/or to detect radiation-induced carcinogenesis so that an appropriate countermeasure administration can be implemented. The techniques are also targeted at non-invasively monitoring cancer patients after the primary cancer has been resected to determine if/when a secondary tumor develops.

### STATEMENT OF GOVERNMENT SUPPORT

This invention was made in part with government support under grant number NPRF00402 (NSBRI NCC 9-58), from the National Aeronautics and Space Administration. The United States government has certain rights to the invention.

### FIELD OF INVENTION

The present invention relates to methods and compositions for the protection and monitoring of diseases and disorders. Disease prophylaxis, development, progression, and/or regression after therapeutic treatments (i.e., for example, radiotherapy), may be monitored by alterations in gene expression profiles. For example, a colorectal disease may be exemplified by colorectal cancer. The initiation and development of colorectal diseases may result from radiation exposure (i.e., for example, such as that resulting from medical therapeutics or environmental sources), wherein specific dietary considerations may provide effective prevention and/or treatment.

### BACKGROUND OF THE INVENTION

Diseases and disorders of the colon, rectum and appendix, collectively referred to as the colorectal region, affect millions of people worldwide. One of the most recognizable diseases, colorectal cancer, is among the most common forms of cancer and a leading cause of cancer-related death in the Western world. Current methods for detecting colorectal cancer and pre-cancerous lesions and polyps are based largely on the use of invasive, tube-based cameras known as colonoscopes or sigmoidoscopes. The use of such devices is often a source of anxiety and extreme discomfort for a patient. Therefore, the development and implementation of non-invasive methods and assays for detecting biomedical indicators or biomarkers associated with colorectal cancer holds great appeal. However, current non-invasive methods lack both the necessary sensitivity of the aforementioned invasive techniques and the capacity for detecting alterations in the expression of genes associated with colorectal cancer.

Thus, there is a need for the development of non-invasive methods for preventing and monitoring the initial occurrence and post-treatment reoccurrence of colorectal diseases and disorders.

### SUMMARY OF THE INVENTION

The present invention relates to methods and compositions for the protection and monitoring of diseases and disorders. Disease prophylaxis, development, progression, and/or regression after therapeutic treatments (i.e., for example, radiotherapy), may be monitored by alterations in gene expression profiles. For example, a colorectal disease may be exemplified by colorectal cancer. The initiation and development of colorectal diseases may result from radiation exposure (i.e., for example, such as that resulting from medical therapeutics or environmental sources), wherein specific dietary considerations may provide effective prevention and/or treatment.

In one embodiment, the present invention contemplates a method, comprising: a) providing; i) exfoliated colonocytes derived from a subject; and ii) a gene expression array; b) extracting nucleic acids from said colonocytes; c) contacting said extracted nucleic acids with said gene expression array under conditions that create a gene expression profile. In one embodiment, the nucleic acids comprise ribonucleic acid. In one embodiment, the ribonucleic acid comprises messenger ribonucleic acid. In one embodiment, the nucleic acids comprise deoxyribonucleic acids. In one embodiment, the exfoliated colonocytes are collected from fecal material In one embodiment, the subject has been exposed to radiation. In one embodiment, the subject has consumed a fish oil/pectin diet. In one embodiment, the gene expression profile comprises down-regulated activators of the Ras-PI3K/Akt signaling pathway. In one embodiment, the gene expression profile comprises up-regulated Wnt/β-catenin pathway genes. In one embodiment, the gene expression profile comprises down-regulated Wnt/β-catenin pathway genes. In one embodiment, the gene expression profile comprises up-regulated post-translational modification genes. In one embodiment, the post-translational modification comprises a chemical modification. In one embodiment, the gene expression profile comprises down-regulated EPH receptor genes. In one embodiment, the gene expression profile comprises down-regulated tyrosine kinase receptor genes. In one embodiment, the gene expression profile comprises up-regulated nuclear translocation genes. In one embodiment, the gene expression profile comprises down-regulated tyrosine kinase pathway genes. In one embodiment, the gene expression profile comprises down-regulated cell adhesion genes. In one embodiment, the gene expression profile comprises down-regulated cell cycle regulator genes. In one embodiment, the gene expression profile comprises down-regulated cell proliferation genes. In one embodiment, the gene expression profile comprises down-regulated signal transduction genes. In one embodiment, the gene expression profile comprises up-regulated tumor suppressor genes. In one embodiment, the gene expression profile comprises down-regulated tumor invasion genes. In one embodiment, the gene expression profile comprises up-regulated mitotic arrest genes.

In one embodiment, the present invention contemplates a method, comprising, a) providing; i) a subject at risk for the development of a colorectal disease; ii) a composition comprising a mixture of fish oil and pectin; b) administering the composition to the subject under conditions such that the colorectal disease development is reduced. In one embodiment, the disease development is prevented. In one embodiment, the subject at risk comprises a genetic predisposition for the colorectal disease. In one embodiment, the colorectal disease or disorder is selected from the group including, but not limited to, colorectal cancer, colon cancer, large bowel cancer, colonic polyps, anal cancer, general anal and rectal diseases, colitis, Crohn's disease, hemorrhoids, ischemic colitis, ulcerative colitis, diverticulosis, diverticulitis and irritable bowel syndrome. In one embodiment, the subject is a human. In one embodiment, the administering comprises dietary consumption. In one embodiment, the cancer development reduction results from increased colorectal cell apoptosis. In one embodiment, the cancer development reduction results from decreased colorectal cell proliferation. In one embodiment, the increased apoptosis comprises an upregulation of genes associated with the Wnt/β-catenin pathways. In one embodiment, the decreased cell proliferation comprises an upregulation of genes associated with the Wnt/β-catenin pathways. In one embodiment, the increased apoptosis comprises a downregulation of tyrosine kinase related genes. In one embodiment, the decreased cell proliferation comprises a downregulation of tyrosine kinase related genes. In one embodiment, the tyrosine kinase related genes regulate Ras-PI3K/Akt pathways. In one embodiment, the subject at risk has been exposed to a carcinogen. In one embodiment, the subject at risk has been exposed to a radiation source. In one embodiment, the radiation source comprises cosmic radiation. In one embodiment, the radiation source comprises a medical device. In one embodiment, the radiation source comprises heavy ion particles. In one embodiment, the radiation source comprises protons. In one embodiment, the heavy ion particles comprise iron. In one embodiment, the radiation source comprises beta particles. In one embodiment, the radiation source comprises gamma rays. In one embodiment, the radiation source comprises X-rays. In one embodiment, the radiation source comprises a radioactive element. In one embodiment, the radioactive element comprises cobalt. In one embodiment, the radioactive element comprises uranium. In one embodiment, the radioactive element comprises plutonium. In one embodiment, the radioactive element comprises thorium.

In one embodiment, the present invention contemplates a method, comprising: a) providing; i) a subject exhibiting at least one symptom of a colorectal disease; ii) a composition comprising a mixture of fish oil and pectin; b) administering the composition to the subject under conditions such that at least one symptom is reduced. In one embodiment, the colorectal disease or disorder is selected from the group including, but not limited to, colorectal cancer, colon cancer, large bowel cancer, colonic polyps, anal cancer, general anal and rectal diseases, colitis, Crohn's disease, hemorrhoids, ischemic colitis, ulcerative colitis, diverticulosis, diverticulitis and irritable bowel syndrome. In one embodiment, the subject is a human. In one embodiment, the administering comprises dietary consumption. In one embodiment, the symptom reduction results from increased colorectal cell apoptosis. In one embodiment, the symptom reduction results from decreased colorectal cell proliferation. In one embodiment, the increased apoptosis comprises an upregulation of genes associated with the Wnt/β-catenin pathways. In one embodiment, the decreased cell proliferation comprises an upregulation of genes associated with the Wnt/β-catenin pathways. In one embodiment, the increased apoptosis comprises a downregulation of tyrosine kinase related genes. In one embodiment, the decreased cell proliferation comprises a downregulation of tyrosine kinase related genes. In one embodiment, the tyrosine kinase related genes regulate Ras-PI3K/Akt pathways. In one embodiment, the cancer development is derived from a carcinogen exposure. In one embodiment, the cancer development is derived from radiation exposure. In one embodiment, the subject has been exposed to a radiation source. In one embodiment, the radiation source comprises cosmic radiation. In one embodiment, the radiation source comprises a medical device. In one embodiment, the radiation source comprises heavy ion particles. In one embodiment, the radiation source comprises protons. In one embodiment, the heavy ion particles comprise iron. In one embodiment, the radiation source comprises beta particles. In one embodiment, the radiation source comprises gamma rays. In one embodiment, the radiation source comprises X-rays. In one embodiment, the radiation source comprises a radioactive element. In one embodiment, the radioactive element comprises cobalt. In one embodiment, the radioactive element comprises uranium. In one embodiment, the radioactive element comprises plutonium. In one embodiment, the radioactive element comprises thorium.

In one embodiment, the present invention contemplates a method, comprising: a) providing; i) a subject comprising a plurality of exfoliated colonocytes; ii) a plurality of nucleic acid samples derived from the plurality of exfoliated colonocytes; iii) a first gene expression profile created from a first nucleic acid sample; iv) a second gene expression sample created from a second nucleic acid sample; b) comparing the first gene expression profile to the second gene expression profile. In one embodiment, the nucleic acids comprise ribonucleic acid. In one embodiment, the ribonucleic acid comprises messenger ribonucleic acid. In one embodiment, the nucleic acids comprise deoxyribonucleic acids. In one embodiment, the exfoliated colonocytes are collected from fecal material. In one embodiment, the subject is diagnosed with a disease. In one embodiment, the disease comprises a colorectal disease. In one embodiment, the subject has been exposed to radiation. In one embodiment, the comparing identifies a disease regression. In one embodiment, the comparing identifies a disease development. In one embodiment, the comparing identifies a disease stasis. In one embodiment, the comparing identifies down-regulated activators of the Ras-PI3K/Akt signaling pathway. In one embodiment, the comparing identifies up-regulated Wnt/β-catenin pathway genes. In one embodiment, the comparing identifies down-regulated Wnt/β-catenin pathway genes. In one embodiment, the comparing identifies up-regulated post-translational modification genes. In one embodiment, the post-translational modification comprises a chemical modification. In one embodiment, the comparing identifies down-regulated EPH receptor genes. In one embodiment, the comparing identifies down-regulated tyrosine kinase receptor genes. In one embodiment, the comparing identifies up-regulated nuclear translocation genes. In one embodiment, the comparing identifies down-regulated tyrosine kinase pathway genes. In one embodiment, the comparing identifies down-regulated cell adhesion genes. In one embodiment, the comparing identifies down-regulated cell cycle regulator genes. In one embodiment, the comparing identifies down-regulated cell proliferation genes. In one embodiment, the comparing identifies down-regulated signal transduction genes. In one embodiment, the comparing identifies up-regulated tumor suppressor genes. In one embodiment, the comparing identifies down-regulated tumor invasion genes. In one embodiment, the comparing identifies up-regulated mitotic arrest genes.

In one embodiment, the present invention contemplates a composition comprising a mixture of fish oil and pectin. In one embodiment, the composition comprises a tablet. In one embodiment, the composition comprises a capsule. In one embodiment, the composition comprises a gel. In one embodiment, the composition further comprises a meat binder. In one embodiment, the composition further comprises a vegetable binder. In one embodiment, the composition further comprises a fruit binder. In one embodiment, the composition comprises a dairy binder.

### BRIEF DESCRIPTION OF THE FIGURES

FIG. 1 illustrates one embodiment of a timeline for radiation exposure, carcinogen injection, and fecal material collection to support a gene expression analysis.

FIG. 2 presents a representative software program capable of creating a Gene Ontology analysis.

FIG. 3 presents exemplary data showing post-translational protein modification pathways identified by Gene Ontology (GO) analysis. Some genes within this GO term are involved in the Wnt/β-catenin pathway, which is believed to promote colon cancer by enhancing proliferation and inhibiting apoptosis

FIG. 4 presents exemplary data showing differential gene expression of the tyrosine kinase pathway in response to dietary factors identified by GO analysis.

FIG. 5 presents one embodiment of a putative countermeasure mechanism for radiation exposure.

FIG. 6 presents one embodiment of an overall experimental design for determining a temporal gene expression analysis of fish oil and pectin diet administration.

FIG. 7 presents one embodiment of fecal matter collection timeline to differentiate between colorectal cancer stage phenotypes.

FIG. 8 presents one embodiment of an RNA isolation/extraction procedure for a GO gene expression analysis.

FIG. 9 presents exemplary data showing that a fish oil/pectin diet results in lower aberrant crypt foci formation and higher apoptotic index compared with corn oil/cellulose following induced colorectal cancer.

FIG. 10 illustrates one embodiment of an upregulated apoptotic pathway in an early stage (ACF) colorectal cancer cell.

FIG. 11 presents exemplary data of a downregulated proliferative pathway in an intermediate stage colorectal cancer cell.

FIG. 12A presents exemplary data of an upregulated apoptotic pathway in a tumor stage colorectal cancer cell.

FIG. 12B presents exemplary data of a downregulated PPAR δ receptor pathway in a tumor stage colorectal cancer cell.

FIG. 13 illustrates one embodiment of an upregulated apoptotic pathway in a tumor stage colorectal cancer cell.

FIG. 14 presents exemplary data showing phenotypic anticarcinogenic representations of fish oil/pectin diets. FP=Fish Oil/Pectin Diet. CC=Corn Oil/Cellulose Diet. R(+)=1 Gy Fe radiation. R(−)=Sham-Irradiation.

FIG. 14A: Abberant crypt foci incidence in promotion stage rats.

FIG. 14B: Tumor incidence in tumor stage rats.

FIG. 15 presents illustrative embodiments of differentially expressed genes that encode proteins that may effect the Wnt signaling pathway subsequent to radiation exposure.

FIG. 15A: Data interpretation from rats exposed to radiation compared with those not exposed to radiation.

FIG. 15B: Data interpretation from rats fed fish oil/pectin diets compared with corn oil/cellulose diets.

FIG. 16 presents exemplary data of FO/P enhanced colonocyte apoptosis as compared to CO/C at 24 hr after AOM injection (P<0.024) The apoptotic index is 100 times the mean number of apoptotic cells per crypt column divided by the total number of cells per crypt column. The data are means±SEM.

FIG. 17 presents exemplary data of dietary changes on cell proliferation and apoptosis. The data are means±SEM.

FIG. 17A: FO/P suppression of HM ACF formation (p=0.0002).

FIG. 17B: Fish oil/pectin reduction of cell proliferation (p=0.0001).

FIG. 17C: FO/P significantly enhanced colonocyte apoptosis compared to CO/C (p=0.027).

FIG. 18 presents exemplary data of dietary changes on tumor incidence and apoptosis. The data are means±SEM.

FIG. 18A: Animal receiving FO/P diet had significantly lower colon tumor incidence. Data was analyzed by Chi-square analysis (p=0.029).

FIG. 18B: FO/P significantly enhanced colonocyte apoptosis compared to CO/C at 31 wk after AOM injection (p=0.027).

FIG. 19 presents exemplary data showing a correlation of fecal and mucosal microarray gene expression levels. Data are expressed as the level of gene expression from FO/P fed rats relative to CO/C fed rats. (R2=0.613)

### DEFINITIONS

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, “colorectal disease” and “colorectal disorder” refer to diseases and disorders of the colon, rectum and appendix. While not limiting the scope of the invention in any way, colorectal diseases and disorders include but are in no way limited to colorectal cancer, colon cancer, large bowel cancer, colonic polyps, anal cancer, general anal and rectal diseases, colitis, Crohn's disease, hemorrhoids, ischemic colitis, ulcerative colitis, diverticulosis, diverticulitis and irritable bowel syndrome.

As used herein, “colorectal cancer”, also known as “colon cancer”, “large rectal cancer” and “anal cancer,” is a disease that originates from the epithelial cells lining the gastrointestinal tract. The disease is often characterized by the cancerous growths residing in the colon, rectum and/or appendix. Symptoms associated with colorectal cancer include but are in no way limited to change in bowel habits, change in the appearance of stool including but not limited to bloody stool, rectal bleeding, stool with mucus, and/or black tar-like stool, bowel obstruction, the presence of an abdominal tumor, unexplained weight loss, jaundice, abdominal pain, anemia and blood clots.

A “colonocyte” refers to an epithelial cell that lines the mammalian colon.

The term “regression” as used herein, refers to any trend or shift toward a lower, less severe state that represents a progressive decline (as in size or severity) of a manifestation of disease (i.e., for example, tumor regression following chemotherapeutics).

The term “development” as used herein, refers to any trend or shift toward a greater, more severe state that represents a progressive increase (as in size or severity) of a manifestation of disease (i.e., for example, tumor growth following carcinogen exposure).

The term “stasis” as used herein refers to a lack of any trend or shift in any state regarding the manifestation of disease.

As used herein, “energy percentage” is the percentage of energy, i.e. calories, derived from a macronutrient, including but in no way limited to carbohydrates, proteins and fats consumed by a subject.

As used herein, the terms “prevent” and “preventing” include the prevention of the recurrence, spread or onset of a disease or disorder. It is not intended that the present invention be limited to complete prevention. In some embodiments, the onset is delayed, or the severity of the disease or disorder is reduced.

As used herein, the terms “treat” and “treating” are not limited to the case where the subject (e.g. patient) is cured and the disease is eradicated. Rather, the present invention also contemplates treatment that merely reduces symptoms, improves (to some degree) and/or delays disease progression. It is not intended that the present invention be limited to instances wherein a disease or affliction is cured. It is sufficient that symptoms are reduced.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, and it can be expressed as the ratio LD50/ED50. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term, “purified” or “isolated”, as used herein, may refer to a peptide composition that has been subjected to treatment (i.e., for example, fractionation) to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to ‘apparent homogeneity” such that there is single protein species (i.e., for example, based upon SDS-PAGE or HPLC analysis). A purified composition is not intended to mean that some trace impurities may remain.

As used herein, the term “substantially purified” refers to molecules, either nucleic or amino acid sequences, that are removed from their natural environment, isolated or separated, and are at least 60% free, preferably 75% free, and more preferably 90% free from other components with which they are naturally associated. An “isolated polynucleotide” is therefore a substantially purified polynucleotide.

The term “biocompatible”, as used herein, refers to any material that does not elicit a substantial detrimental response in the host. There is always concern, when a foreign object is introduced into a living body, that the object will induce an immune reaction, such as an inflammatory response that will have negative effects on the host. In the context of this invention, biocompatibility is evaluated according to the application for which it was designed: for example; a bandage is regarded a biocompatible with the skin, whereas an implanted medical device is regarded as biocompatible with the internal tissues of the body. Preferably, biocompatible materials include, but are not limited to, biodegradable and biostable materials.

“Nucleic acid sequence” and “nucleotide sequence” as used herein refer to an oligonucleotide or polynucleotide, and fragments or portions thereof, and to DNA or RNA of genomic or synthetic origin, which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

As used herein the term “portion” when in reference to a protein (as in “a portion of a given protein”) refers to fragments of that protein. The fragments may range in size from four amino acid residues to the entire amino acid sequence minus one amino acid.

The term “portion” when used in reference to a nucleotide sequence refers to fragments of that nucleotide sequence. The fragments may range in size from 5 nucleotide residues to the entire nucleotide sequence minus one nucleic acid residue.

The term “biologically active” refers to any molecule having structural, regulatory or biochemical functions. For example, biological activity may be determined, for example, by restoration of wild-type growth in cells lacking protein activity. Cells lacking protein activity may be produced by many methods (i.e., for example, point mutation and frame-shift mutation). Complementation is achieved by transfecting cells that lack protein activity with an expression vector which expresses the protein, a derivative thereof, or a portion thereof.

Low stringency conditions comprise conditions equivalent to binding or hybridization at 42° C. in a solution consisting of 5×SSPE (43.8 g/l NaCl, 6.9 g/l NaH2PO4.H2O and 1.85 g/l EDTA, pH adjusted to 7.4 with NaOH), 0.1% SDS, 5× Denhardt's reagent {50× Denhardt's contains per 500 ml: 5 g Ficoll (Type 400, Pharmacia), 5 g BSA (Fraction V; Sigma)} and 100 μg/ml denatured salmon sperm DNA followed by washing in a solution comprising 5×SSPE, 0.1% SDS at 42° C. when a probe of about 500 nucleotides in length is employed. Numerous equivalent conditions may also be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions that promote hybridization under conditions of high stringency (e.g., increasing the temperature of the hybridization and/or wash steps, the use of formamide in the hybridization solution, etc.) may also be used.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C0 t or R0 t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tm” is used in reference to the “melting temperature.” The melting temperature is the temperature at which a population of double-stranded nucleic acid molecules becomes half dissociated into single strands. As indicated by standard references, a simple estimate of the Tm value may be calculated by the equation: Tm=81.5+0.41 (% G+C), when a nucleic acid is in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tm.

As used herein the term “stringency” is used in reference to the conditions of temperature, ionic strength, and the presence of other compounds such as organic solvents, under which nucleic acid hybridizations are conducted. “Stringency” typically occurs in a range from about Tm to about 20° C. to 25° C. below Tm. A “stringent hybridization” can be used to identify or detect identical polynucleotide sequences or to identify or detect similar or related polynucleotide sequences. For example, when fragments are employed in hybridization reactions under stringent conditions the hybridization of fragments which contain unique sequences (i.e., regions which are either non-homologous to or which contain less than about 50% homology or complementarity) are favored. Alternatively, when conditions of “weak” or “low” stringency are used hybridization may occur with nucleic acids that are derived from genetically diverse organisms (i.e., for example, the frequency of complementary sequences is usually low between such organisms).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of a target sequence of interest. In contrast, “background template” is used in reference to nucleic acid other than sample template, which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

“Amplification” is defined as the production of additional copies of a nucleic acid sequence and is generally carried out using polymerase chain reaction. Dieffenbach C. W. and G. S. Dveksler (1995) In: PCR Primer, a Laboratory Manual, Cold Spring Harbor Press, Plainview, N.Y.

As used herein, the term “polymerase chain reaction” (“PCR”) refers to the method of K. B. Mullis U.S. Pat. Nos. 4,683,195 and 4,683,202, herein incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxy-ribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers; to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “restriction endonucleases” and “restriction enzymes” refer to bacterial enzymes, each of which cut double-stranded DNA at or near a specific nucleotide sequence.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence that encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, etc.

The term “in operable combination” as used herein, refers to any linkage of nucleic acid sequences in such a manner that a nucleic acid molecule capable of directing the transcription of a given gene and/or the synthesis of a desired protein molecule is produced. Regulatory sequences may be operably combined to an open reading frame including but not limited to initiation signals such as start (i.e., ATG) and stop codons, promoters which may be constitutive (i.e., continuously active) or inducible, as well as enhancers to increase the efficiency of expression, and transcription termination signals.

Transcriptional control signals in eukaryotes comprise “promoter” and “enhancer” elements. Promoters and enhancers consist of short arrays of DNA sequences that interact specifically with cellular proteins involved in transcription. Maniatis, T. et al., Science 236:1237 (1987). Promoter and enhancer elements have been isolated from a variety of eukaryotic sources including genes in plant, yeast, insect and mammalian cells and viruses (analogous control elements, i.e., promoters, are also found in prokaryotes). The selection of a particular promoter and enhancer depends on what cell type is to be used to express the protein of interest.

The presence of “splicing signals” on an expression vector often results in higher levels of expression of the recombinant transcript. Splicing signals mediate the removal of introns from the primary RNA transcript and consist of a splice donor and acceptor site. Sambrook, J. et al., In: Molecular Cloning: A Laboratory Manual, 2nd ed., Cold Spring Harbor laboratory Press, New York (1989) pp. 16.7-16.8. A commonly used splice donor and acceptor site is the splice junction from the 16S RNA of SV40.

The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is isolated from one gene and placed 3′ of another gene. Efficient expression of recombinant DNA sequences in eukaryotic cells involves expression of signals directing the efficient termination and polyadenylation of the resulting transcript. Transcription termination signals are generally found downstream of the polyadenylation signal and are a few hundred nucleotides in length.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “Southern blot” refers to the analysis of DNA on agarose or acrylamide gels to fractionate the DNA according to size, followed by transfer and immobilization of the DNA from the gel to a solid support, such as nitrocellulose or a nylon membrane. The immobilized DNA is then probed with a labeled oligodeoxyribonucleotide probe or DNA probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists. J. Sambrook et al. (1989) In: Molecular Cloning: A Laboratory Manual, Cold Spring Harbor Press, NY, pp 9.31-9.58.

The term “Northern blot” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled oligodeoxyribonucleotide probe or DNA probe to detect RNA species complementary to the probe used. Northern blots are a standard tool of molecular biologists. J. Sambrook, J. et al. (1989) supra, pp 7.39-7.52.

The term “reverse Northern blot” as used herein refers to the analysis of DNA by electrophoresis of DNA on agarose gels to fractionate the DNA on the basis of size followed by transfer of the fractionated DNA from the gel to a solid support, such as nitrocellulose or a nylon membrane. The immobilized DNA is then probed with a labeled oligoribonucleotide probe or RNA probe to detect DNA species complementary to the ribo probe used.

As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

As used herein, the term “structural gene” refers to a DNA sequence coding for RNA or a protein. In contrast, “regulatory genes” are structural genes that encode products which control the expression of other genes (e.g., transcription factors).

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences that direct the termination of transcription, posttranscriptional cleavage and polyadenylation.

### DETAILED DESCRIPTION OF THE INVENTION

The present invention relates to methods and compositions for the protection and monitoring of diseases and disorders. Disease prophylaxis, development, progression, and/or regression after therapeutic treatments (i.e., for example, radiotherapy), may be monitored by alterations in gene expression profiles. For example, a colorectal disease may be exemplified by colorectal cancer. The initiation and development of colorectal diseases may result from radiation exposure (i.e., for example, such as that resulting from medical therapeutics or environmental sources), wherein specific dietary considerations may provide effective prevention and/or treatment.

### I. Radiation Exposure and Cancer Development

Safe, long duration, manned space flight has long been questioned because of the high risk for exposure to ionizing radiation and the resultant increased risk for later cancer development. Durante et al., “Heavy ion carcinogenesis and human space exploration” Nat Rev Cancer 8:465-472 (2008). Of all the cancers, colon cancer appears susceptible to radiation enhancement since it is the second-leading cause of cancer deaths and effects men and women essentially equally. Also, it has been reported that gastrointestinal tract cell linings may be susceptible to radiation-induced DNA damage. Turner et al., “Opportunities for nutritional amelioration of radiation-induced cellular damage” Nutrition 18:904-912 (2002). Further, airline pilots who frequently fly long trips and are repeatedly exposed to higher levels of radiation have been associated with a higher incidence of colon cancer than the general public. Rafnsson et al., “Incidence of cancer among commercial airline pilots” Occup Environ Med 57:175-179 (2000). Studies in which rats or mice are treated with a combination of a colon specific carcinogen and ionizing radiation exposure show a synergistic effect on tumor incidence as compared to chemical carcinogen alone. Tanaka et al., “Synergistic effect of radiation on colon carcinogenesis induced by methylazoxymethanol acetate in ACI/N rats” Jpn J Cancer Res 84:1031-1036 (1993); and Sharp et al., “Apparent synergism between radiation and the carcinogen 1,2-dimethylhydrazine in the induction of colonic tumors in rats” Radiat Res 117:304-317 (1989).

Space radiation comprises ionizing radiation that can exist as heavy ion radiation in a form of high mass, high atomic number and high energy ions (HZE). HZEs can be carcinogenic by various pathways including, but not limited to, i) inducing a loss of cell cycle checkpoint control; ii) inducing DNA lesions; iii) activating oncogenes such as Ras family, cMyc and/or NF-kB; and/or iv) inhibiting tumor suppressor genes like p53 to cause cell cycle delay. Durante et al., “Heavy ion carcinogenesis and human space exploration” Nat Rev Cancer 8:465-472 (2008). HZE interaction with biological cells may result in not only physical damage to DNA but also may impart oxidative damage to DNA via the formation of free radicals, which are well documented to be carcinogenic. Ionizing radiation also increases levels of reactive oxygen species (ROS) directly and indirectly from the radiolysis of water, damaging mitochondrial DNA and inducing genomic instability with oxidative damage, enhances the rate of mutation and genetic changes in descendants of the irradiated cells. These mechanisms may contribute toward carcinogenesis, and colon cancer is a likely candidate for radiation enhancement. Turner et al., “Opportunities for nutritional amelioration of radiation-induced cellular damage” Nutrition 18:904-912 (2002).

### II. Colorectal Cancer

Colon cancer continues to be the second highest contributor to cancer deaths in the United States. Jemal et al., “Cancer Statistics 2009” CA Cancer J Clin. 59:225-249 (2009). It has been reported that up to 80% of colon cancers may be preventable by dietary intervention Cummings et al., “Diet and the prevention of cancer” BMJ. 317:1636-1640 (1998). Diets containing a combination of fish oil (FO), which is rich in n-3 polyunsaturated fatty acids (PUFAs), and pectin (P), a highly fermentable fiber that yield high levels of butyrate upon microflora fermentation, resulted in lower colon tumor incidence than a diet containing the combination of corn oil and cellulose (CO/C). Chang et al., “Predictive value of proliferation, differentiation and apoptosis as intermediate markers for colon tumorigenesis” Carcinogenesis 721-730 (1997). Corn oil is rich in the n-6 PUFA, and cellulose is a fiber with very low extent of fermentation. One of the mechanisms by which FO/P may be protective against colon cancer is the induction of apoptosis, a programmed cell death that plays a role in sustaining cell homeostasis by allowing the removal of damaged cells. Chang et al., “Predictive value of proliferation, differentiation and apoptosis as intermediate markers for colon tumorigenesis” Carcinogenesis 18:721-730 (1997); Hong et al., “Dietary fish oil reduces O6-methylguanine DNA adduct levels in rat colon in part by increasing apoptosis during tumor initiation” Cancer Epidemiol Biomarkers Prev. 9:819-26 (2000); and Vanamala et al., “Dietary fish oil and pectin enhance colonocyte apoptosis in part through suppression of PPARdelta/PGE2 and elevation of PGE3” Carcinogenesis 29:790-796 (2008). Tumor development depends not only on suppression of apoptosis, but also on increased cell proliferation. It has been reported that a FO/P diet also suppresses cell proliferation relative to a CO/C diet. Chang et al., “Fish oil blocks azoxymethane-induced rat colon tumorigenesis by increasing cell differentiation and apoptosis rather than decreasing cell proliferation” J Nutr. 128:491-497 (1998).

The development of colon cancer in both humans and rats is believed to be a multi-step process and may comprise both genetic and gene expression profiles that change over time. First, an inactivation of the adenomatous polyposis coli (APC) tumor-suppressor gene may result in high levels of the Wnt signaling pathway molecule β-catenin. Although it is not necessary to understand the mechanism of an invention, it is believed that β-catenin can then enter the nucleus thereby driving an inappropriate activation of target gene transcription rates and/or promoting the development of colon tumorigenesis. Bienz et al., “Linking colorectal cancer to Wnt signaling” Cell 103:311-320 (2000). Consistent with this hypothesis are observations that up-regulation of the Wnt pathway increased intracellular β-catenin levels and nuclear translocation as a consequence of radiation-induced colorectal cancer. Nakashima et al., “Altered expression of beta-catenin during radiation-induced colonic carcinogenesis” Pathol Res Pract 198:717-724 (2002). Furthermore, it is believed that genetic aberrations giving rise to excessive Wnt pathway signaling may be present in approximately 70-80% of colorectal cancers. Consequently, the Wnt-β-catenin pathway may play a role in clinical chemoprevention strategies.

Early detection of colorectal cancer can greatly increase the prognosis for a subject exhibiting symptoms associated with the disease, thus it is desirable to have accurate screening methods and assays. Rutter et al., “Thirty-year analysis of a colonoscopic surveillance program for neoplasia in ulcerative colitis” Gastroenterology 130(4):1030-1038. (2006). Consistent with this goal, the adoption of non-invasive methodology designed to reduce anxiety over colorectal cancer screening and improve overall acceptance of the screening process would be highly desirable. Unfortunately, current non-invasive detection methods lack sensitivity and are incapable of detecting alterations in gene expression. Moreover, once an individual has received treatment for colon disease, there is always a chance of recurrence.

A non-invasive methodology that would permit routine screening of these patients would reduce anxiety associated with recurrence between invasive screenings and permit more frequent testing to provide early detection of the recurrent disease. Whenever therapy for any disease includes radiation exposure, there is always a chance of secondary tumors within the colon, as colon epithelial cells are susceptible to the damage induced by radiation exposure. Therefore, use of a gene expression technique would also facilitate monitoring for colon cancer development in all patients exposed to radiotherapy that would generate likely damage within the abdomen.

In one embodiment, the present invention contemplates a method comprising a gene expression profile capable of identifying regulatory mechanisms that either promote, or prevent, the development of colorectal diseases and disorders (i.e., for example, colorectal cancer). Thus, the present invention utilizes a novel, non-invasive methodology based on the analysis of fecal or stool samples, which contain intact sloughed colon cells (i.e., for example, exfoliated colonocytes), in order to quantify colorectal disease and disorder relevant gene expression profiles.

In one embodiment, the present invention contemplates a method comprising a noninvasive technique in which intact eukaryotic mRNA are isolated from exfoliated colonocytes to monitor gene expression profiles. Davidson et al., “Noninvasive detection of putative biomarkers for colon cancer using fecal messenger RNA” Cancer Epidemiol Biomarkers Prev. 4:643-647 (1995); and Davidson et al., “Non-invasive detection of fecal protein kinase C betaII and zeta messenger RNA: putative biomarkers for colon cancer” Carcinogenesis 19:253-257 (1998). In one embodiment, the method further comprises determining gene expression profiles. In one embodiment, the gene expression profile identifies modulation of apoptosis. In one embodiment, the gene expression profile identified modulation of cell proliferation. In one embodiment, the gene expression profiles are derived from exfoliated colonocyte messenger ribonucleic acid (mRNA). In one embodiment, the mRNA is collected at least three colon tumorigeneic stages including, but not limited to: i) initiation stage; ii) aberrant crypt foci (ACF) stage; and iii) tumor stage. In one embodiment, fecal microarray data are compared with phenotypic data to establish the regulatory controls contributing to the chemoprotective effects of a fish oil/pectin diet.

A. Exfoliated Colonocytes

The present invention provides for novel, non-invasive methodologies utilizing feces, which contain exfoliated colonocytes, in order to quantify colonic mRNAs. Davidson et al. (1995) Cancer Epidemiology Biomarkers and Prevention 4, 643-647; Davidson et al. (1998) Carcinogenesis 19, 253-257; Davidson et al. (2003) Biomarkers 8, 51-61, all of which are hereby incorporated by reference. Approximately one-sixth to one-third of normal adult colonic epithelial cells are shed daily. Potten, Biochimica et Biophysica Acta 560, 281-299 (1979), herein incorporated by reference.

Although RNA is generally less suitable than DNA because it is readily degraded, it has previously been demonstrated that intact fecal eukaryotic mRNA can be isolated because of the presence of viable exfoliated colonocytes in the fecal stream. Albaugh (1992) International Journal of Cancer 52, 347-350; Davidson et al. (1995) Cancer Epidemiology Biomarkers and Prevention 4, 643-647; Davidson et al. (2003) Biomarkers 8, 51-61; Santiago et al. (2003) Journal of Virology 77, 2233-2242 and Kanaoka et al. (2004) Gastroenterology 127, 422-427, all of which are incorporated herein by reference.

Using exfoliated colonocytes, mRNA expression signatures have been reported to discriminate between conditions associated with inflammatory bowel disease versus normal conditions, as well as conditions consistent with the presence of adenoma versus normal conditions. Davidson et al., Biomarkers 8, 51-61 (2003). This report suggested that mRNA isolated from exfoliated human colonocytes can be used to detect early stages of colon cancer, and possibly chronic inflammation.

### II. Gene Expression Profiles

Until the present disclosure, a useful microarray gene expression profile-based classification of colonic diseases for diagnostic purposes was unavailable. In one embodiment, the present invention contemplates using non-invasive mRNA procedures in patients at high risk for colorectal adenoma development and/or recurrence.

In one embodiment, the present invention contemplating identifying diagnostic gene sets (i.e., for example, a single unique gene or a plurality of unique genes) to facilitate an objective classification of different colorectal cancer phenotypes. For example, these methods allow for the identification of both single genes and gene combinations for distinguishing polyps and exposure to a fish oil/pectin diet. For example, the disclosed methods may further reduce the classification error rate, with two and three-gene combinations providing robust classifiers that non-invasively identify discriminative signatures for diagnostic purposes.

A. Fecal Sample Preparation

Some advantages of using fecal samples include, but are not limited to, processing within two hours of excretion, coding versatility, storing at −80 degrees C. for later analysis, and easily isolating poly A⁺ RNA. Davidson et al. (1995) Cancer Epidemiology Biomarkers and Prevention 4, 643-647; Davidson et al. (1998) Carcinogenesis 19, 253-257; Davidson et al. (2003) Biomarkers 8, 51-61, all of which are hereby incorporated by reference. Poly A⁺ RNA isolation procedures usually result in a pure mammalian RNA population, due to potential interference from bacterial RNA contamination. Davidson et al. (1995) Cancer Epidemiology Biomarkers and Prevention 4, 643-647. In addition, an Agilent 2100 Bioanalyzer may used to assess integrity of mucosal and fecal poly A⁺ RNA. Samples can then be processed using a CodeLink™ Gene Expression Assay manual (Applied Microarray, Tempe, Ariz.) and analyzed using the Human whole Genome Expression Bioarray. Davidson et al. (2004) Cancer Research 64, 6797-6804, hereby incorporated by reference. Each array contains the entire human genome derived from publicly available, well-annotated mRNA sequences. This platform is unique because it is capable of detecting minimal differences in gene expression, as low as 1.3-fold with 95% confidence. The 3-D gel provides support for 30-mers in a matrix that holds the probe away from the surface of the slide. This method substantially reduces background and enhances sensitivity, allowing for the detection of one transcript per cell with 50-200 ng of poly A⁺ RNA.

B. Microarray Evaluation

The microarray data presented herein shows that radiation exposure influenced gene expression responsible for post translational modifications. Post-translational protein modifications play a role in carcinogenesis, because chemical modifications of regulatory proteins result in the activation of certain cellular signaling pathways, such as enhanced proliferation, and suppression of cell division or death. Krueger et al., “Posttranslational protein modifications: current implications for cancer detection, prevention, and therapeutics” Mol Cell Proteomics 5:1799-1810 (2006). Furthermore, some of the affected post translational modification genes might play a role in Wnt signaling pathway, which has been implicated in colon carcinogenesis. In particular, the data suggest that radiation exposure may enhance the Wnt signaling pathway and promote colon carcinogenesis.

It has been reported that environmental signals might play a role in colon cancer by inducing nuclear β-catenin accumulation (i.e., for example, translocation from intracellular spaces) and/or specific target gene expression. Based on the microarray data, genes believed to be positive regulators of the Wnt/β-catenin signaling pathway were up-regulated in radiation-induced tumors, thereby suggesting an activation of β-catenin. The data also show that radiation increases high multiplicity aberrant crypt foci formation as compared to AOM injections. Although it is not necessary to understand the mechanism of an invention, it is believed that radiation exposure induces an increase in cell proliferation and/or decreases in apoptosis.

cDNA microarrays may be used to define gene profiles in order to assess how genes are expressed between radiation exposed groups and different dietary intake groups. cDNA microarrays provide a platform to evaluate multiple genes at a time, thereby allowing screening of differential gene expression. A number of screening tools are publicly available: i) DAVID has distinct strengths for the data mining; ii) Gene Ontology (GO) performs enrichment analysis and systematically clusters an abundance of genes to an associated biological annotation. In some embodiments, these techniques allow the identification of overrepresented gene expression pathways following radiation exposure and dietary intake.

Microarrays can be evaluated by inspecting for spot morphology. Marginal spots can be flagged as either background contamination (C) or irregular shape (I) in the output of the scanning software. Spots that pass quality control standards might then be categorized as “good” (G). In addition, spots marked with (L) would indicate a corresponding reading of “near the background”. The low (L) measurements reflect either true low gene expression levels or may have been caused by degradation of the mRNA resulting in a low signal. It has been reported that samples collected from colonic mucosa previously exhibited a relatively low proportion (5-8%) of L spots. Davidson et al. (2004) Cancer Research 64, 6797-6804, incorporated herein by reference. In contrast, the proportion of L spots in data obtained from fecal samples was significantly higher (65-83%).

The standard procedure for microarray data analysis usually involves a normalization step to facilitate the comparison of gene expression levels from two or more arrays. The goal of such a processing step is to reduce the technical variance while preserving the biologically meaningful variance produced by the different experimental conditions/treatments. The normalization procedures can be either “local” or “global”. Quackenbush (2002) Nature Genetics Supplement 32, 496-501, incorporated in its entirety by reference. Other parametric or non-parametric normalization procedures have also been reported. Kerr et al. (2001) Genetic Research 77, 123-128; Sidorov et al. (2002) Information Sciences 146, 65-71; Bolstad et al. (2003) Bioinformatics 19, 185-193, all of which are incorporated herein by reference.

However, none of these previously disclosed methods were developed for the situations where one deals with a high percentage of partially degraded mRNA in the samples. Recently, a two-stage normalization procedure for such data sets was presented. Liu et al. (2005) Bioinformatics 21, 4000-4006, incorporated herein by reference. The method is built on non-parametric smoothing techniques with robustness consideration, and was used to evaluate the feasibility of properly extracting information from fecal mRNA data.

To evaluate the presently disclosed data, however, the main objective of the two-stage normalization is to “regularize” the G spots for each gene while including the L spots that behave “similarly” to other G probes for that same gene, and excluding the outlying G probes. This process involves identifying groups of genes/features that distinguish or classify between the different combinations of risk factors. Although it is not necessary to understand the mechanism of an invention, it is believed that the presently disclosed methods provide a conservative approach that does not include a normalization step, and focuses on a subset of genes that have been implicated in colorectal carcinogenesis. It is further believed that this method is justified by the observation that applying any kind of normalization to a data set with a high percentage of L spots has the potential to “flatten” the signal that results in a loss of data.

C. Identification of Gene Sets

When there is high percentage of L spots on each array in a data set, the spots may be examined for the values of the parameters used by the CodeLink® scanning software and their effect on the number of G spots that are common for a subset of the arrays. To be specific, denoted by A^{k}ⱼ the set of genes xᵢ that have at most j raw mean spot intensity values less than μ_{i,l}+kσ_{i,l} where μ_{i,l} is the value of local background median for the spot representing the gene xᵢ on the lth array, and σ_{i,l} is the corresponding standard deviation for that background signal. For example, A^{1.5}₀ is the set of G spots that are common for all of the arrays in the data set (by default k=1.5 in the CodeLink software). Spots that are flagged C are not considered when the sets A^{k}ⱼ are formed. Notice that A^{k}ⱼ ⊂ A^{s}ᵣ if s≦k and j≦r. In particular, A^{k}ⱼ ⊂ A^{s}ⱼ, s≦k represents the fact that one gets a lesser number of common good spots if one requires a stronger signal as compared to the background. Also, A^{k}ⱼ ⊂ A^{k}ᵣ, j≦r represents the fact that the number of common genes increases if one allows more L spots per gene.

One goal is to determine if mRNA data from fecal colonocytes has the potential to classify different colon cancer risk factors. To this end, a combination of sets A^{k}ⱼ with a set B totaling approximately 1300 known human colonic markers may be used. Using such a priori biological knowledge, sets of common genes for A^{k}ⱼ and B may be investigated.

Based on these results, the intersection A²₁ ∪ B may be determined. This conservative approach provides a subset of the known colonic biomarkers that have strong signal (k=2 compare to the CodeLink weaker default condition k=1.5) and no more than 1 low signal spot on the entire data set. One should notice that the microarray data could be grouped into various combinations of two different classes.

This may be exemplified with an experimental design which examines the effect of radiation exposure at three time points: 7 Weeks, 14 Weeks, and 28 Weeks, and two diets: an experimental diet (i.e., for example, fish oil/pectin) and a control diet (i.e., for example, corn/cellulose). These different groupings produce their respective sets A^{k}ⱼ that could be larger or smaller depending on which of the microarrays are included in the corresponding groups and classes. Obviously, A^{k}ⱼ has the smallest possible size when one considers all of the data as being divided into two major categories, e.g. (+IR) vs (−IR). The next step in finding feature sets is to design classifiers that categorize samples based on the expression values of the genes from the intersection A²₁ ∪ B. An important consideration is that the number of genes in such gene feature sets should be sufficiently small, for example, by constructing the classifiers for feature sets of size 1, 2, and 3. It is believed that classifiers should involve small numbers of genes because: i) the limited number of samples often available in clinical studies makes classifier design and error estimation problematic for large feature sets; and ii) small gene sets facilitate design of practical immunohistochemical diagnostic panels. Thus, a simple linear discriminant analysis (LDA) classifier and a small number of genes are useful with this method.

Given a set of features on which to base a classifier, one has to address not only the classifier design from sample data, but also the estimation of its error. When the number of potential feature sets is large, the key issue is whether a particular feature set provides good classification. A key concern is the precision with which the error of the designed classifier estimates the error of the optimal classifier. When data are limited, an error estimator may have a large variance and therefore may often be low. This can produce many feature sets and classifiers with low error estimates. Algorithms are available that mitigates this problem by applying a bolstered error estimation. Braga-Neto et al. (2004) Pattern Recognition 37, 1267-1281, incorporated in its entirety by reference. Although it is not necessary to understand the mechanism of an invention, it is believed that bolstered error estimation has advantages with respect to commonly used error estimators such as re-substitution, cross-validation, and bootstrap methods for error estimation in terms of speed and accuracy (bias and variance). For example, the method bolsters the original empirical distribution of the available data by means of suitable bolstering kernels placed at each datapoint location. The error can be computed analytically in some cases, such as in the case of LDA. Therefore, a relatively small size of the set A²₁ ∪ B can allow for a comparing the errors of the potential feature sets of size 1, 2, and 3.

### III. Gene Expression Regulation in Carcinogenesis

While colon cancer is believed to be the second-leading cause of cancer mortality in the US for men and women combined, it is also considered to be a condition amenable to diet intervention. In one embodiment, the present invention contemplates a method comprising, providing a composition comprising a fish oil/pectin diet, wherein the composition reduces and/or prevents the development of colon cancer. In one embodiment, the composition reduces and/or prevents colon cancer development as compared to a corn oil/cellulose diet.

Phenotypic data is presented herein showing that radiation treatment increased a proliferative index and decreased an apoptotic index in ACF stage cells. Pectin intake (alone) induced the levels of apoptosis compared to cellulose intake (data not shown). In contrast, in tumor stage cells, a fish oil/pectin diet enhanced apoptosis irrespective of radiation treatment. By performing immunoblot and immunohistochemical analysis for β-catenin, fish oil/pectin diet reduced β-catenin levels in rats administered only AOM, or given radiation exposure and AOM. Vanamala et al., “Dietary fish oil and pectin enhance colonocyte apoptosis in part through suppression of PPARdelta/PGE2 and elevation of PGE3” Carcinogenesis 29:790-796 (2008).

A. Dietary Effects on Cell Proliferation

Fish oil/pectin (FP) diets are believed to protect against colon cancer, as compared to corn oil/cellulose (CC), in part by upregulating apoptosis at the promotion stage of carcinogenesis and/or suppressing peroxisome proliferator-activated receptor δ (PPARδ) expression at the tumor stage. Vanamala et al., “Dietary fish oil and pectin enhance colonocyte apoptosis in part through suppression of PPARδ/PGE2 and elevation of PGE3” Carcinogenesis 29:790-796 (2008). Although it is not necessary to understand the mechanism of an invention, it is believed that the underlying mechanisms of protective FP diets may induce apoptosis and suppress peroxisome proliferator-activated receptor signaling pathways (i.e., for example, PPARδ signals).

A diet high in fish oil (i.e., for example, a source of n-3 fatty acids) and pectin (i.e., for example, a highly fermentable fiber producing butyrate) has been shown to have a protective effect against chemically-induced colon cancer when compared to a corn oil rich (i.e., for example, a source of n-6 fatty acids) and cellulose (i.e., for example, a poorly fermentable fiber) diet. Chang et al., “Fish oil blocks azoxymethane-induced rat colon tumorigenesis by increasing cell differentiation and apoptosis rather than decreasing cell proliferation” J Nutr 128:491-497 (1998). Although it is not necessary to understand the mechanism of an invention, it is believed that the above chemoprotective diet enhanced apoptotic removal of DNA damaged cells. Hong et al., “Fish oil decreases oxidative DNA damage by enhancing apoptosis in rat colon” Nutr Cancer 52:166-175 (2005). It is further believed that the chemoprotective diet suppressed the Wnt/β-catenin signaling pathway at tumor stage as assessed by measuring the level of nuclear β-catenin through immunohistochemistry. Vanamala et al. “Dietary fish oil and pectin enhance colonocyte apoptosis in part through suppression of PPARδ/PGE₂ and elevation of PGE₃. Carcinogenesis 29:790-796 (2008). GO analysis of gene expression profiles identified enriched biological processes a FP diet. See, Table 1.

TABLE 1
Biological Processes Effected By A Fish Oil/Pectin Diet

| GO term - Biological Process | Down-regulated | Up-regulated | P-value |
|---|---|---|---|
| Sensory perception | Accn3, Crybb3, Gabrr2, Gnat1, Grm3, | F2r, Tas2r7 | 0.0010 |
| | Gucy2d, Htr2c, Olr1468, Olr1654, Tacstd2 | | |
| Metal ion transport | Accn3, Atp7b, Glp1r, Kcnab1, Kcnh7, | F2r | 0.010 |
| | Kctdl4, Ndufa9, Ppp3ca, Sfxn5, Slc25a37, | | |
| | Slc39z4, Slc39z6, Slc8a1 | | |
| Cyclic nucleotide mediated signaling | Adcy7, Eif4ebp2, Glp1r, Gnat1, Grm3, | Avpr2 | 0.034 |
| | Htr2c | | |
| Cell surface receptor linked signal | Adcy7, Avpr2, Cd47, Cd97, Eif4ebp2, | Calm2, | 0.039 |
| transduction | Ephb1, F2r, Gabrr2, Gfra3, Glp1r, Gnat1, | Gpr37i1, | |
| | Grm3, Hbp1, Hipk3, Htr2c, Ltbp2, | Tas2r7 | |
| | Map3k7, Met, Olr1468, Olr1654, Ret, | | |
| | Rgs3, Src, Tacstd2, Wnt2b | | |
| Amino sugar metabolic process | Glp1r, Slc8a1 | F2r | 0.041 |
| G-protein coupled receptor protein | Adcy7, Cd97, Gabrr2, Glp1r, Gnat1, Grm3, | Avpr2, | 0.041 |
| signaling pathway | Htr2c, Olr1468, Olr1654 | Calm2, F2r, | |
| | | Gpr37i1, | |
| | | Rgs3, Tas2r7 | |

In general, the GO gene expression analysis isolated factors showing that a fish oil/pectin diet downregulates expression of genes related to tyrosine kinase signal transduction on the cell surface, activators of the Ras-PI3K/Akt signaling.

B. Radiation Effects on Gene Expression

In one embodiment, the present invention contemplates a composition comprising a fish oil/pectin (FP) diet. In one embodiment, the present invention contemplates a method comprising administering an FP diet, wherein the FP diet protects against radiation-enhanced colon carcinogenesis. In one embodiment, the FP diet protection is compared to a corn oil/cellulose (CC) diet. Although it is not necessary to understand the mechanism of an invention, it is believed that such radioprotection may be mediated by maintaining an elevated level of apoptosis.

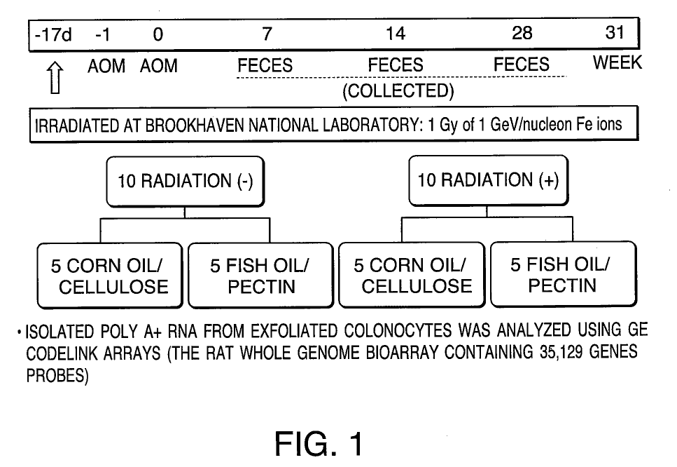

The basic experimental paradigm for the data described herein comprise rats were either sham-irradiated or exposed to radiation (+/−1 Gy, 1 GeV/nucleon Fe ions). On the tenth and seventeenth day following irradiation, the rats were injected with a colon-specific carcinogen (i.e., for example, azoxymethane; AOM: 5 mg/kg BW, SC) and then divided into two groups that were fed diets containing either FP or CC. Fecal material was then collected on Week 7, Week 14, and Week 28. See, FIG. 1.

Using the above paradigm, the data presented herein describes gene expression profiles measured in exfoliated colonocytes following administration of a colorectal cancer protective diet (i.e., for example, fish oil/pectin), and a colorectal cancer promotive diet (i.e., for example, corn oil/cellulose). The effect of diet was analyzed by comparing gene expression patterns from rat exfoliated colonocyte mRNA following radiation exposure and AOM injections

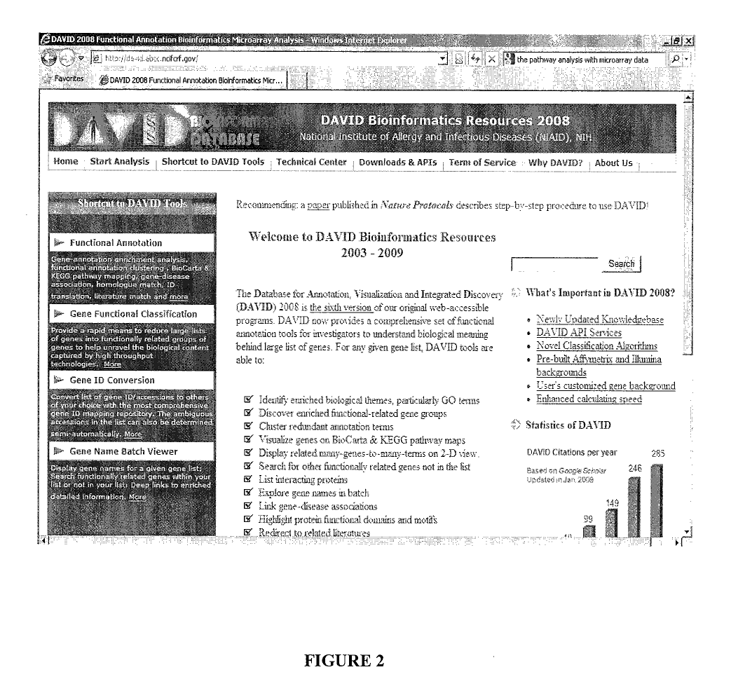

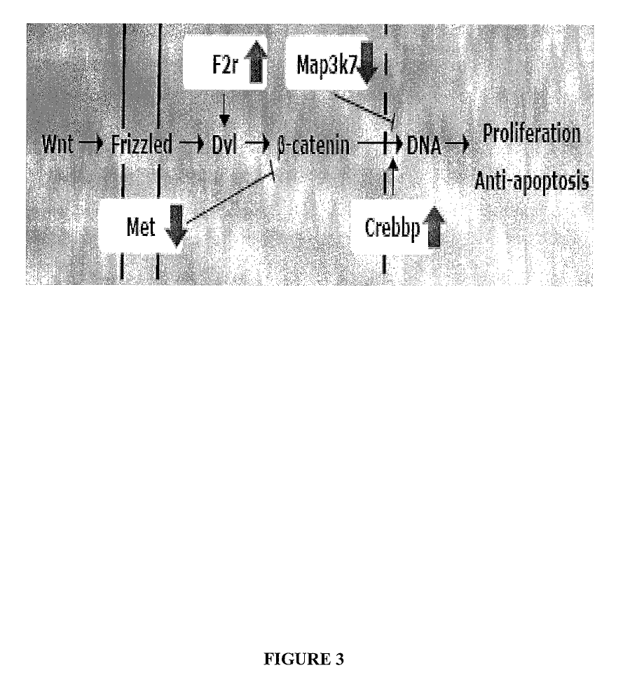

Microarray data were normalized and analyzed using a mixed model ANOVA procedure, and genes exhibiting differential expression underwent Gene Ontology (GO) enrichment analyses (supra). The data was further analyzed utilizing the publicly available DAVID gene expression analysis platform. See, FIG. 2, david.abcc.ncifcrf/gov. DAVID performs a Gene Oncology (GO) analysis and determines a biological process enrichment p-value (e.g., reflected by an increased number of either upregulated genes or downregulated genes). Radiation exposure was found to differentially express approximately one-hundred and twenty-five (125) genes. The DAVID GO analysis identified several groups that were either up-regulated or down-regulated by radiation exposure. See, Table 2.

TABLE 2
Biological Processes Affected By Radiation

| GO term - Biological Process | Down-regulated | Up-regulated | P-value |
|---|---|---|---|
| Sensory perception | Accn3, Htr2c, Tacstd2, Tas2r7 | F2r, Gnat1, Gucy2d | 0.00096 |
| Neurological system process | Accn3, Ap3m2, Htr2c, Tacstd2, | Cldn5, F2r, Gnat1, | 0.022 |
| | Tas2r7 | Gucy2d | |
| Post-translational protein | Arih1, Dusp5, Ephb1, Map3k7, | Crebbp, Bre, F2r, | 0.029 |
| modification | Met, Pctk3, Ppp3ca, Ube2q | Gucy2d, Ube2g1, Vrk3 | |
| Cell surface receptor linked signal | Ephb1, Gpr37i1, Hbp1, Htr2c, | Adcy7, Eif4ebp1, F2r, | 0.036 |
| transduction | Map3k7, Met, Tacstd2, Tas2r7 | Gnat1, Rgs3 | |

GO analysis identified several genes involved in post-translational protein modification. For example, some genes within this GO term are involved in the Wnt/β-catenin pathway, which promotes colon cancer by enhancing cell proliferation and inhibiting apoptosis. This particular gene expression pattern, suggests that one effect of radiation exposure may enhance colon cancer by up-regulating a Wnt/β-catenin pathway. Although it is not necessary to understand the mechanism of an invention, it is believed that Wnt/β-catenin pathway may be involved in cell proliferation and apoptosis.

C. Dietary Effects on Gene Expression Modulation of Carcinogenesis

Most chemoprevention studies have been targeted at understanding the benefits derived from interventions at a single point in the carcinogenic process. In one embodiment, the present invention contemplates a method for monitoring chemoprotection provided by a FO/P diet. In one embodiment, the chemoprotection occurs at carcinogenesis initiation. In one embodiment, the chemoprotection occurs at carcinogenesis promotion. In one embodiment, the chemoprotection occurs a carcinogenesis tumor development. In one embodiment, the method uses a noninvasive technique that permits the isolation of eukaryotic mRNA from exfoliated colonocytes in fecal material. In one embodiment, gene expression was monitored using microarray procedures, wherein the resulting data were compared with phenotypic data to determine if the gene expression profiles identified by Gene Ontology analysis were predictive of changes in disease phenotypes.

In one embodiment, the present invention contemplates a method comprising modulating differential gene expression involved in apoptosis and/or cell proliferation related to colon carcinogenesis using a dietary composition. In one embodiment, the dietary composition is a fish oil/pectin composition. In one embodiment, the dietary composition is a corn oil/cellulose composition. In one embodiment, the differentially expressed genes are identified at the carcinogenic initiation stage. In one embodiment, the differentially expressed genes are identified at the carcinogenic promotion stage. Although it is not necessary to understand the mechanism of an invention, it is believed that the expression of many more genes were affected by diet at the promotion stage versus the initiation stage, suggesting this stage is more susceptible to the FO/P dietary intervention. For example, Gene Oncology (GO) pathways enriched at this stage include both cell proliferation and apoptosis functions. In one embodiment, the differentially expressed genes are identified at the tumor development stage. Although it is not necessary to understand the mechanism of an invention, it is believed that the dietary effects on tumor stage gene expression pathways are principally associated with apoptosis functions.

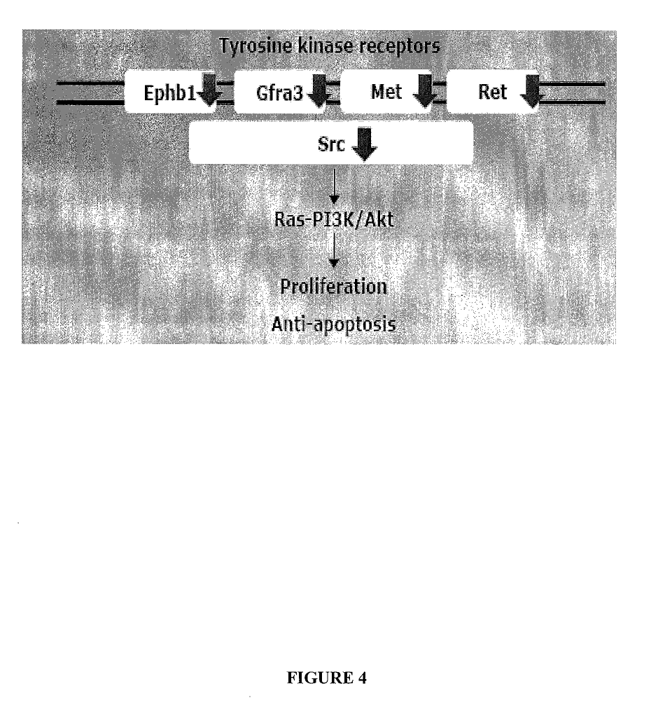

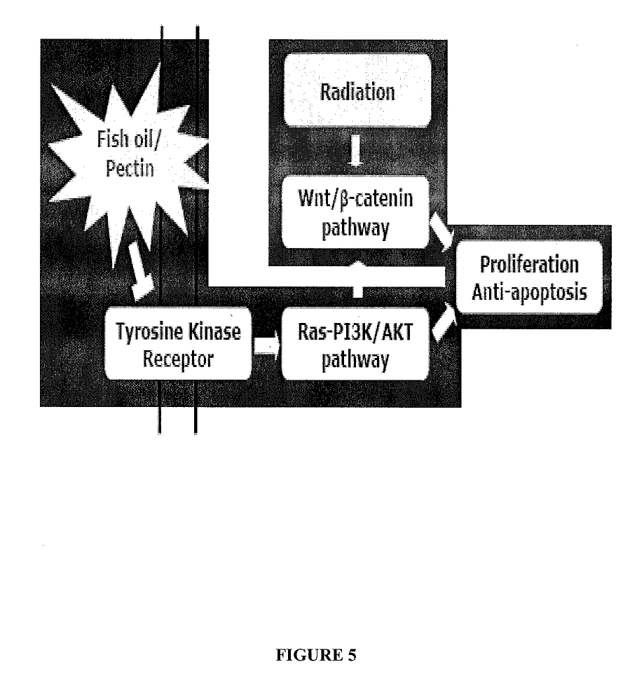

Gene expression profiles described above suggested that dietary modifications affected genes associated with cell surface receptor linked signal transduction: For example, the fish oil/pectin diet down-regulates expression of genes related to tyrosine kinase signal transduction on the cell surface, wherein the tyrosine kinase pathway is believed to be an activator of Ras-PI3K/Akt signaling. See, FIG. 4. These data suggest that fish oil/pectin diet may suppress colon cancer via down-regulation of gene expression related to tyrosine kinase signaling pathway.

It has been suggested that colon cancer might be prevented through changing diets. The chemopreventive activity of fish oil containing the n-3 fatty acids, eicosapentaenoic acid (EPA, 20:5n-3) and docosahexaenoic acid (DHA, 22:6n-3) on colon carcinogenesis has been reported in many studies. For example, fish oil intake decreased alkylating and oxidative DNA damage by increasing apoptosis. Hong et al., “Fish oil decreases oxidative DNA damage by enhancing apoptosis in rat colon” Nutr Cancer 52:166-175 (2005).

Pectin is a highly fermentable fiber that is degraded by microorganisms in the colon to short-chain fatty acids such as butyrate. Butyrate has been suggested to prevent colon cancer by inhibiting the enzyme histone deacetylase (HDAC) and induce the cyclin-dependent kinase inhibitor p21/Cip/WAF1. Lupton J. R., “Microbial degradation products influence colon cancer risk: the butyrate controversy” J Nutr 134:479-482 (2004).

Four combinations of two kinds of fat and dietary fiber has been reported to provide a synergistic carcinogenic protective effect. In particular, a combination of fish oil and pectin reduced colon tumor incidence and initiated apoptosis. Although it is not necessary to understand the mechanism of an invention, it is believed that the colon tumor reduction was due to enhanced unsaturation of mitochondrial phospholipids by generating reactive oxygen species (ROS) and by recruiting a Ca²⁺ dependent mitochondrial intrinsic pathway as compared to a corn oil/cellulose diet. Chang et al., “Fish oil blocks azoxymethane-induced rat colon tumorigenesis by increasing cell differentiation and apoptosis rather than decreasing cell proliferation” J Nutr 128:491-497 (1998); Hong et al., “Fish oil increases mitochondrial phospholipid unsaturation, upregulating reactive oxygen species and apoptosis in rat colonocytes” Carcinogenesis 23:1919-1925 (2002); and Kolar et al., “Docosahexaenoic acid and butyrate synergistically induce colonocyte apoptosis by enhancing mitochondrial Ca²⁺ accumulation” Cancer Res 67:5561-5568 (2007). Although it is not necessary to understand the mechanism of an invention, it is believed that a comparison of two diets, a cancer promotive corn oil/cellulose diet and cancer preventive fish oil/pectin diet, may be detectable using gene expression analysis.

GO gene expression analysis isolated factors showing that a fish oil/pectin diet down-regulates some tyrosine kinase related genes, which are regulators of the Ras-PI3K/Akt signaling pathway. Although it is not necessary to understand the mechanism of an invention, it is believed that the Ras-PI3K/Akt signaling pathway may promote cell proliferation and suppress apoptosis.

Consequently, the data suggest that FP dietary factors may counterbalance some radiation-induced effects on cell proliferation and apoptosis, thereby preventing the occurrence and/or development of cancer, for example, by reducing radiation affected expression of genes within the Wnt/β-catenin pathways, which would promote colon cancer by enhancing proliferation and suppressing apoptosis. On the other hand, a fish oil/pectin diet reduced expression of genes related to tyrosine kinase, which would suppress colon carcinogenesis by inhibiting PI3K/Akt enhancement of proliferation and suppression of apoptosis. Because gene profiling using fecal material is a non-invasive method, certain embodiments may be practiced in environments where sophisticated medical facilities are not readily available (i.e., for example, to monitor astronaut health during long duration space flight).

The above data suggest that fish oil/pectin (FP) diets are protective against colon cancer as compared to corn oil/cellulose (CC). Although it is not necessary to understand the mechanism of an invention, it is believed that this protective effect may be, in part, a result of upregulating apoptosis at the promotion stage of carcinogenesis and suppressing peroxisome proliferator-activated receptor δ (PPAR δ) expression at the tumor stage. To elucidate the underlying mechanisms whereby FP diets induce apoptosis and suppress PPAR signals, temporal gene expression profiles from exfoliated rat colonocytes were analyzed.

It has also been shown that in azoxymethane (AOM)-injected rats, the colon mucosal expression of cytosolic β-catenin, cyclin D₁ and/or wnt2 was significantly elevated after a corn oil diet but suppressed after a fish oil diet. Fujise et al., “Long-term feeding of various fat diets modulates azoxymethane-induced colon carcinogenesis through Wnt/beta-catenin signaling in rats” Am J Physiol Gastrointest Liver Physiol 292:G1150-1156 (2007). Consistent with these observations is that butyrate may attenuate transcription of c-myc and cyclin D1 genes, which may be target genes of the Wnt/β-catenin signaling pathway in colon carcinogenesis. Maier et al., “Butyrate and vitamin D3 induce transcriptional attenuation at the cyclin D1 locus in colonic carcinoma cells” J Cell Physiol 218:638-642 (2009).

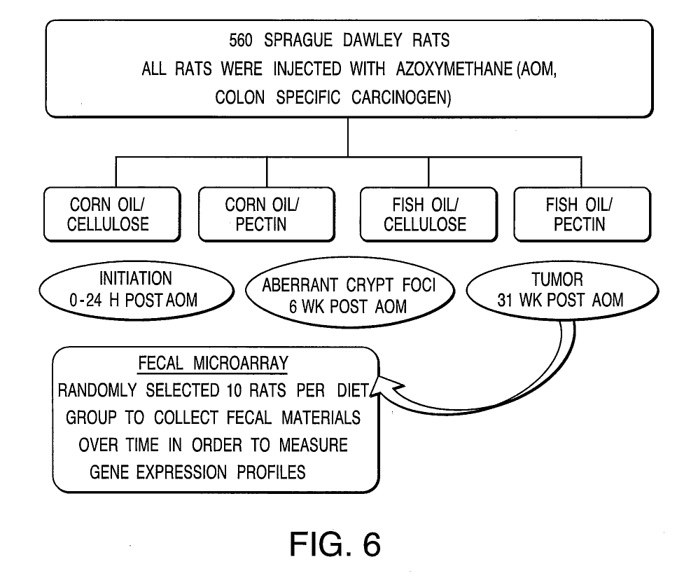

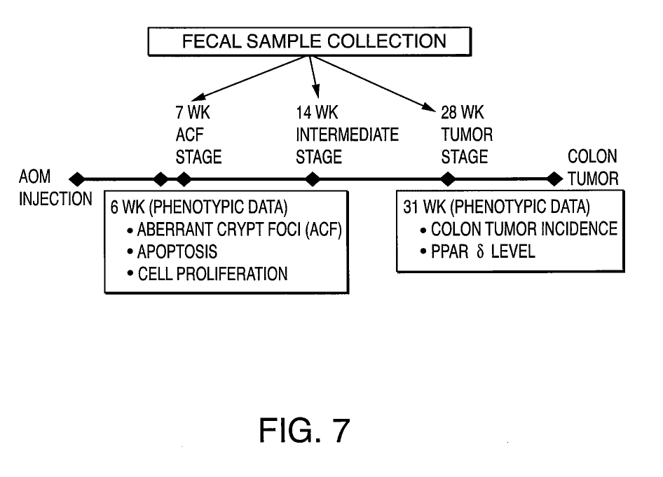

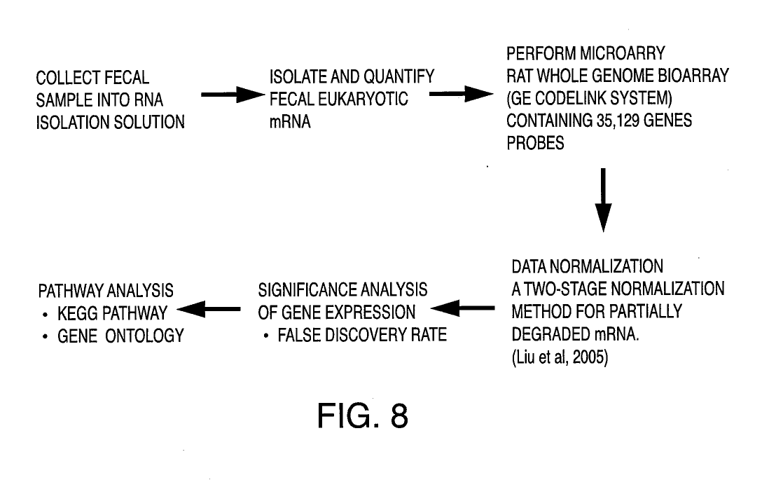

Consequently, some data presented herein was collected after azoxymethane injection (AOM; 2×, 15 mg/kg BW, sc) in combination with consumption of either a FP or CC diet. See, FIG. 6. A subset of rats were randomly selected for fecal matter collection at Week 7, Week 14, and Week 28 following AOM injection. See, FIG. 7. The fecal material was processed and poly A+ RNA was extracted from colonocytes. See, FIG. 8. This paradigm generally resulted in cancer initiation within twenty-four (24) hours after AOM injection, followed by aberrant crypt formation within six (6) weeks, while overt tumor appearance occurred within approximately thirty-one (31) weeks.

1. Initiation Stage

The data indicated that at least three (3) annotated genes were differentially expressed between the fish oil/pectin and corn oil/cellulose diets that are consistent with previous reports. Davidson et al., “Chemopreventive n-3 polyunsaturated fatty acids reprogram genetic signatures during colon cancer initiation and progression in the rat” Cancer Research 64:6797-804 (2004). Several potential explanations for this phenomenon exist, one of which is the dramatic shift in tissue homeostasis that occurs in response to AOM exposure. In addition to these three annotated genes, six (6) non-annotated genes were differentially expressed in colonocytes after AOM injection as a result of diet. See Table 3.

TABLE 3
Differentially expressed genes in response to FO/P diet compared with CO/C at
initiation stage

| | Gene | | Relative | |
|---|---|---|---|---|
| GenBank accession | symbol | Gene name | expression¹ | Function |
| AI502557 | B4galt1 | udp-gal:betaglcnac beta 1,4- | 0.37 | cell adhesion |
| | | galactosyltransferase, polypeptide 1 | | |
| NM_001002835 | Smoc1 | sparc-related modular calcium binding | 0.60 | positive regulation of |
| | | protein 1 | | cell-substrate |
| | | | | adhesion |
| CB726708 | Scarb2 | scavenger receptor class B, member 2 | 0.05 | cell adhesion |
| BI281095 | NULL | UI-R-DD0-bzr-b-09-0-UIs1 UI-R-DD0 | 2.96 | |
| | | cDNA clone UI-R-DD0-bzr-b-09-0-UI | | |
| | | 3′ | | |
| BF404993 | NULL | UI-R-CA1-bio-b-08-0-UIs1 UI-R-CA1 | 0.47 | |
| | | cDNA clone UI-R-CA1-bio-b-08-0-UI | | |
| | | 3′ | | |
| BF398623 | NULL | UI-R-BS2-ber-a-01-0-UIs1 UI-R-BS2 | 0.64 | |
| | | cDNA clone UI-R-BS2-ber-a-01-0-UI | | |
| | | 3′ | | |
| BF523734 | NULL | UI-R-Y0-vb-g-07-0-UIr1 UI-R-Y0 | 0.41 | |
| | | cDNA clone UI-R-Y0-vb-g-07-0-UI 5′ | | |
| | | AMGNNUC:MRPE3-00069-A3-A | | |
| CB738608 | NULL | placenta embryo D17 (10379) cDNA | 0.57 | |
| | | clone mrpe3-00069-a3 5′ | | |
| BF403189 | NULL | UI-R-CA0-bhs-g-02-0-UIs1 UI-R-CA0 | 0.12 | |
| | | cDNA clone UI-R-CA0-bhs-g-02-0-UI | | |
| | | 3′ | | |

¹Relative expression was calculated by FO/P divided by CO/C expression level. Less than 1.00 indicates that the relative gene expression level was lower in rats consuming FO/P diet compared to CO/C.

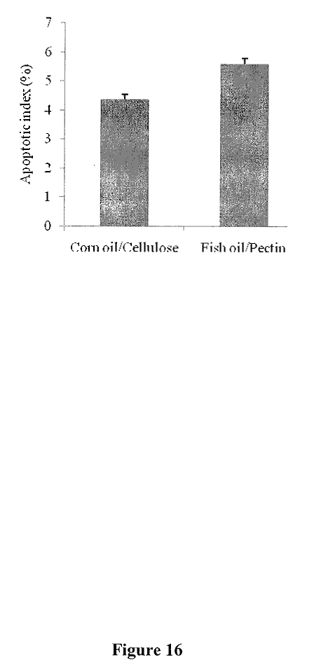

The relative levels of expression for the three annotated genes were lower in rats consuming a diet containing FO/P as compared to CO/C and all three are involved in maintaining cell adhesion. It has been reported that cell adhesion to basement membranes may prevent cell death, suggesting that the FO/P diet would facilitate apoptosis induction. Stupack et al., “Get a ligand, get a life: integrins, signaling and cell survival” J Cell Sci. 115:3729-3738 (2002). To determine if the FO/P diet effect on expression of cell adhesion genes was related to the occurrence of cell death, apoptosis was measured in rat colon. Although there were no changes in the expression of genes involved in apoptosis 24 hours after AOM injection, the lower expression of cell adhesion genes in FO/P rats did correspond to an enhancement in apoptosis at this time. See, FIG. 16.

However, analysis of rat colon tissues demonstrated differences in apoptosis between the diets, with FO/P enhancing apoptosis (p=0.024). The three annotated differentially expressed genes between the two diet groups are associated with cell adhesion. Although it is not necessary to understand the mechanism of an invention, it is believed that the lower relative expression of cell adhesion promoters in cells after AOM injection likely contributes to the ability of FO/P rats to effectively eliminate cells with DNA damage. Whether the elimination is through the induction of apoptosis or by cell sloughing, these changes in gene expression would explain some of the chemoprotection provided by a FO/P diet, relative to the effects observed with a CO/C diet. Stupack et al., “Get a ligand, get a life: integrins, signaling and cell survival” J Cell Sci. 115:3729-3738 (2002); and Chao et al., “Colorectal cancer cell adhesion attenuates Ad-E2F-1 mediated apoptosis” J Surg Res. 113:81-87 (2003).

2. ACF Stage

Changes in the expression of genes involved in cell cycle regulation have been reported to be associated with the promotion of colon carcinogenesis. Polyak et al., “Early alteration of cell-cycle-regulated gene expression in colorectal neoplasia” Am J Pathol. 149:381-387 (1996). At the ACF stage, the data described below show that a FO/P diet resulted in altered gene expression profiles annotated to cell cycle related categories

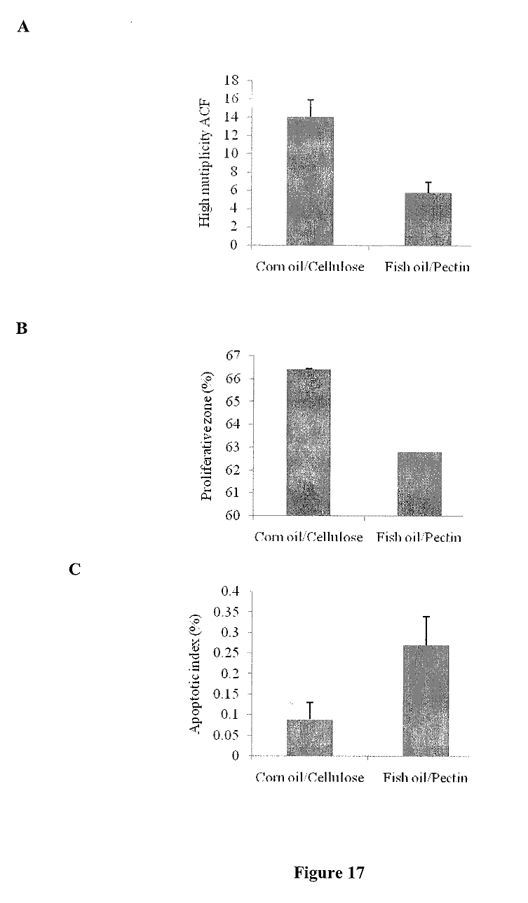

FO/P suppression of the formation of early preneoplastic lesions of colon cancer (aberrant crypts) was measured by the number of HM ACF, an indicator of later colon tumor development. Rats receiving the FO/P diet had fewer HM ACF than rats receiving the CO/C diet See, FIG. 17A. Colonocyte proliferation and apoptosis were also determined, wherein relative to observations from rats consuming the CO/C diet, rats consuming FO/P diet had an elevated apoptotic index and smaller proliferative zone. See, FIG. 17B and FIG. 17C, respectively.

Fecal microarray analysis detected six hundred and two (602) genes that were differentially expressed between the diets. Upon completion of a GO analysis, eighty (80) biological process categories were significantly enriched. See, Table 4.

TABLE 4
GO terms significantly overrepresented in the genes altered by the fish oil/pectin diet
compared to the corn oil/cellulose diet at the ACF stage

| | | | Total | |
|---|---|---|---|---|
| | | Genes | genes | |
| GO number | GO term (biological process) | in list | in list | PValue |
| GO: 0016043 | cellular component organization and biogenesis | 81 | 2340 | 2.16E−06 |
| GO: 0047496 | vesicle transport along microtubule | 4 | 6 | 1.73E−04 |
| GO: 0043283 | biopolymer metabolic process | 105 | 3698 | 2.56E−04 |
| GO: 0006996 | organelle organization and biogenesis | 36 | 931 | 5.11E−04 |
| GO: 0044238 | primary metabolic process | 160 | 6310 | 5.12E−04 |
| GO: 0022402 | cell cycle process | 21 | 445 | 1.10E−03 |
| GO: 0065007 | biological regulation | 107 | 3929 | 1.10E−03 |
| GO: 0006366 | transcription from RNA polymerase II promoter | 24 | 547 | 1.16E−03 |
| GO: 0043170 | macromolecule metabolic process | 139 | 5405 | 1.19E−03 |
| GO: 0007049 | cell cycle | 23 | 518 | 1.31E−03 |
| GO: 0007018 | microtubule-based movement | 9 | 103 | 1.42E−03 |
| GO: 0009893 | positive regulation of metabolic process | 19 | 398 | 1.81E−03 |
| GO: 0006810 | Transport | 69 | 2333 | 1.97E−03 |
| GO: 0051179 | Localization | 79 | 2761 | 2.02E−03 |
| GO: 0051234 | establishment of localization | 71 | 2420 | 2.03E−03 |
| GO: 0031325 | positive regulation of cellular metabolic process | 18 | 371 | 2.10E−03 |
| GO: 0051649 | establishment of cellular localization | 31 | 841 | 2.89E−03 |
| GO: 0030705 | cytoskeleton-dependent intracellular transport | 9 | 118 | 3.33E−03 |
| GO: 0045941 | positive regulation of transcription | 15 | 292 | 3.42E−03 |
| GO: 0008088 | axon cargo transport | 4 | 15 | 3.42E−03 |
| GO: 0051641 | cellular localization | 31 | 851 | 3.44E−03 |
| GO: 0022607 | cellular component assembly | 21 | 491 | 3.47E−03 |
| GO: 0006139 | nucleobase, nucleoside, nucleotide and nucleic acid | 75 | 2656 | 3.99E−03 |
| | metabolic process | | | |
| GO: 0050789 | regulation of biological process | 93 | 3447 | 4.09E−03 |
| GO: 0065009 | regulation of a molecular function | 20 | 464 | 4.11E−03 |
| GO: 0043412 | biopolymer modification | 46 | 1449 | 4.23E−03 |
| GO: 0045935 | positive regulation of nucleobase, nucleoside, | 15 | 307 | 5.25E−03 |
| | nucleotide and nucleic acid metabolic process | | | |
| GO: 0008090 | retrograde axon cargo transport | 3 | 6 | 6.19E−03 |
| GO: 0065003 | macromolecular complex assembly | 19 | 448 | 6.24E−03 |
| GO: 0006464 | protein modification process | 44 | 1403 | 6.53E−03 |
| GO: 0016568 | chromatin modification | 8 | 106 | 6.81E−03 |
| GO: 0006357 | regulation of transcription from RNA polymerase II | 18 | 424 | 7.95E−03 |
| | promoter | | | |
| GO: 0044237 | cellular metabolic process | 152 | 6267 | 8.20E−03 |
| GO: 0006461 | protein complex assembly | 13 | 262 | 9.19E−03 |
| GO: 0051276 | chromosome organization and biogenesis | 13 | 263 | 9.45E−03 |
| GO: 0006950 | response to stress | 34 | 1034 | 9.69E−03 |
| GO: 0006325 | establishment and/or maintenance of chromatin | 11 | 202 | 1.01E−02 |
| | architecture | | | |
| GO: 0031323 | regulation of cellular metabolic process | 52 | 1777 | 1.08E−02 |
| GO: 0046907 | intracellular transport | 24 | 658 | 1.10E−02 |
| GO: 0000074 | regulation of progression through cell cycle | 13 | 270 | 1.15E−02 |
| GO: 0006323 | DNA packaging | 11 | 207 | 1.19E−02 |
| GO: 0043687 | post-translational protein modification | 37 | 1171 | 1.22E−02 |
| GO: 0051726 | regulation of cell cycle | 13 | 273 | 1.22E−02 |
| GO: 0008152 | metabolic process | 167 | 7051 | 1.23E−02 |
| GO: 0045893 | positive regulation of transcription, DNA- | 12 | 240 | 1.23E−02 |
| | dependent | | | |
| GO: 0045449 | regulation of transcription | 46 | 1541 | 1.25E−02 |
| GO: 0050790 | regulation of catalytic activity | 17 | 412 | 1.31E−02 |
| GO: 0043549 | regulation of kinase activity | 11 | 212 | 1.38E−02 |
| GO: 0006350 | transcription | 48 | 1642 | 1.51E−02 |
| GO: 0007010 | cytoskeleton organization and biogenesis | 18 | 455 | 1.51E−02 |
| GO: 0009100 | glycoprotein metabolic process | 8 | 125 | 1.60E−02 |
| GO: 0006259 | DNA metabolic process | 21 | 571 | 1.68E−02 |
| GO: 0006351 | transcription, DNA-dependent | 44 | 1486 | 1.69E−02 |
| GO: 0007242 | intracellular signaling cascade | 39 | 1280 | 1.69E−02 |
| GO: 0051338 | regulation of transferase activity | 11 | 219 | 1.70E−02 |
| GO: 0032774 | RNA biosynthetic process | 44 | 1489 | 1.74E−02 |
| GO: 0051704 | multi-organism process | 11 | 224 | 1.96E−02 |
| GO: 0019219 | regulation of nucleobase, nucleoside, nucleotide | 46 | 1595 | 2.18E−02 |
| | and nucleic acid metabolic process | | | |
| GO: 0009101 | glycoprotein biosynthetic process | 7 | 106 | 2.38E−02 |
| GO: 0019222 | regulation of metabolic process | 52 | 1863 | 2.47E−02 |
| GO: 0050794 | regulation of cellular process | 79 | 3052 | 2.57E−02 |
| GO: 0018193 | peptidyl-amino acid modification | 7 | 109 | 2.68E−02 |
| GO: 0006355 | regulation of transcription, DNA-dependent | 41 | 1407 | 2.71E−02 |
| GO: 0010468 | regulation of gene expression | 47 | 1663 | 2.78E−02 |
| GO: 0006914 | autophagy | 3 | 13 | 2.92E−02 |
| GO: 0045859 | regulation of protein kinase activity | 10 | 206 | 2.94E−02 |
| GO: 0000079 | regulation of cyclin-dependent protein kinase | 4 | 34 | 3.36E−02 |
| | activity | | | |
| GO: 0032502 | developmental process | 72 | 2778 | 3.39E−02 |
| GO: 0019538 | protein metabolic process | 80 | 3138 | 3.44E−02 |
| GO: 0045944 | positive regulation of transcription from RNA | 9 | 179 | 3.46E−02 |
| | polymerase II promoter | | | |
| GO: 0030518 | steroid hormone receptor signaling pathway | 4 | 35 | 3.62E−02 |
| GO: 0043406 | positive regulation of MAP kinase activity | 5 | 60 | 3.67E−02 |
| GO: 0018108 | peptidyl-tyrosine phosphorylation | 5 | 61 | 3.86E−02 |
| GO: 0016070 | RNA metabolic process | 50 | 1837 | 4.13E−02 |
| GO: 0018212 | peptidyl-tyrosine modification | 5 | 63 | 4.27E−02 |
| GO: 0048522 | positive regulation of cellular process | 29 | 954 | 4.30E−02 |
| GO: 0030001 | metal ion transport | 15 | 403 | 4.37E−02 |
| GO: 0006512 | ubiquitin cycle | 11 | 261 | 4.80E−02 |
| GO: 0048518 | positive regulation of biological process | 31 | 1050 | 4.97E−02 |
| GO: 0048519 | negative regulation of biological process | 30 | 1009 | 4.99E−02 |

Among the eighty (80) clusters, five were directly involved with cell cycles: i) GO:0007049, cell cycle; ii) GO:0022402, cell cycle process; iii) GO:0000074, regulation of progression through cell cycle; iv) GO:0051726, regulation of cell cycle; and v) GO:0000079, regulation of cyclin-dependent protein kinase activity. The cell cycle category (GO:0007049) was selected as representative and selected for a more detailed study. FO/P diets yielded almost uniform lower levels of expression of both cell cycle promoters and suppressors in the cell cycle category (GO:0007049). See, Table 5.

TABLE 5
Relative expression of genes in the cell cycle category (GO: 0007049)

| | | | |
|---|---|---|---|
| | | | Relative |
| GenBank Accession | Gene symbol | Description | expression¹ |

Cell cycle promoters

| | | | |
|---|---|---|---|
| BF388494 | App | amyloid beta (a4) precursor protein | 0.40 |
| NM_012923 | Ccng1 | Cyclin G1 | 0.44 |
| BI294914, | Ccnk | Cyclin K | 0.69 |
| BE113451 | | | |
| BQ206043 | Ccnl1 | Cyclin l1 | 0.31 |
| AI411332 | Ccnt2_predicted | Cyclin T2 (predicted) | 0.55 |
| AA819214 | Cdc34_predicted | Cell division cycle 34 homolog (S. cerevisiae) | 0.51 |
| | | (predicted) | |
| AW532478 | Gfi1b_predicted | Growth factor independent 1B | 0.64 |
| | | (predicted) | |
| NM_053347 | Nde1 | Nuclear distribution gene E homolog | 0.54 |
| | | 1 (A nidulans) | |
| AA685941 | Pafah1b1 | Platelet-activating factor | 0.56 |
| | | acetylhydrolase, isoform ib, alpha | |
| | | subunit 45 kda | |
| NM_012801 | Pdgfa | Platelet derived growth factor, alpha | 0.50 |
| BI395817 | Ruvbl1 | ruvb-like protein 1 | 0.56 |

Cell cycle suppressors

| | | | |
|---|---|---|---|
| NM_001005902 | Abtb1 | Ankyrin repeat and BTB (POZ) | 0.40 |
| | | domain containing 1 | |
| AY351678 | Cdkn1c | Cyclin-dependent kinase inhibitor 1C | 0.48 |
| | | (P57) | |
| BF406173 | Ddit3 | DNA-damage inducible transcript 3 | 0.46 |
| BQ191258 | Dst_predicted | Dystonin (predicted) | 0.39 |
| NM_053484 | Gas7 | Growth arrest specific 7 | 0.49 |
| NM_032080 | Gsk3b | Glycogen synthase kinase 3 beta | 0.53 |
| AW916463.1 | Pms2_predicted | Postmeiotic segregation increased 2 | 3.23 |
| | | (S. cerevisiae) (predicted) | |
| AW526814 | Rbl2 | Retinoblastoma-like 2 (p130) | 0.61 |
| NM_031745.2 | Clip1 | CAP-GLY domain containing linker | 0.32 |
| | | protein 1 | |
| BI297192, | Spin1 | Spindlin 1 | 0.41 |
| NM_001024796.1 | | | |

¹Relative expression was calculated by FO/P divided by CO/C expression level. Less than 1.00 indicates that the relative gene expression level was lower in rats consuming FO/P diet compared to CO/C. All genes have p-value less than 0.05, indicating that genes were differentially expressed by diet.

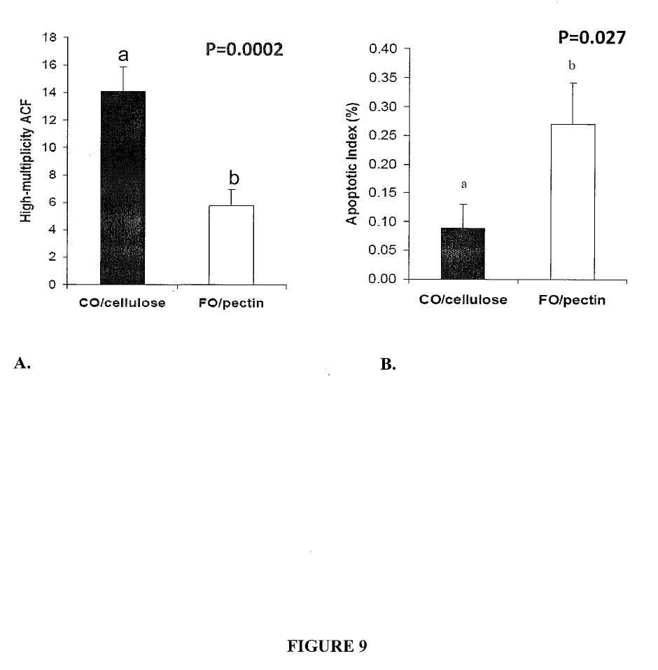

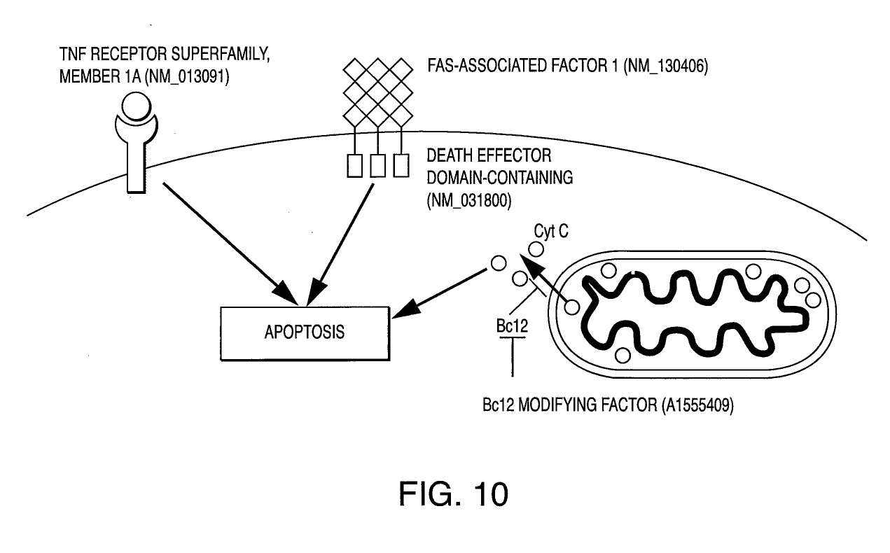

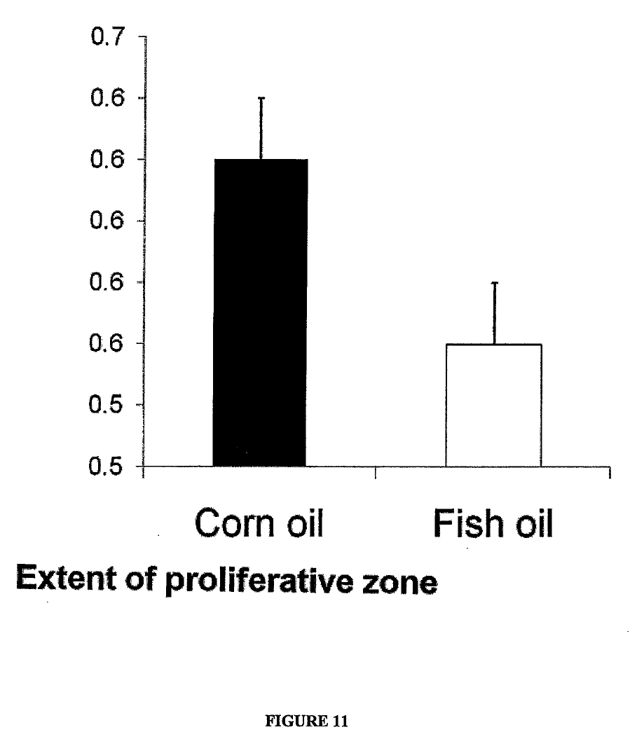

Seven (7) weeks after AOM injection colorectal cancer enters the ACF stage. The data show that a fish oil/pectin diet resulted in lower aberrant crypt foci formation and higher apoptotic index as compared with a corn oil/cellulose diet. See, FIG. 9A and FIG. 9B, respectively. Fecal microarray GO analysis indicated an upregulation enrichment in the apoptosis pathway due to up-regulation of genes by fish oil/pectin; GO category: GO:0006915; Number of genes: twenty-five (25); p<0.008. For example, some of these upregulated apoptosis pathway genes include, but are not limited to: i) TNF member superfamily member 1A (NM_{—}13091); ii) Fas-associated factor 1 (NM_{—}130406); iii) death-effector domain-containing (NM_{—}091800); or iv) Bc12 modifying factor (AI1555409). One possible pathway for their concerted interaction is provided. See, FIG. 10.

Cyclins are believed to be oncogenes that may regulate the cell cycle in that cyclins may interact with cyclin dependent kinase (CDKs) and facilitates phosphorylation of Rb12/p130. Expression of cyclin G1, K, L1, T2, and cdc34 are lower in rats consuming the FO/P diet as compared to expression in a corn oil/cellulose diet. Cdc34 is believed to be involved with p4OSIC1 ubiquitination, a potent CDK inhibitor. Eliseeva et al., “Expression and localization of the CDC34 ubiquitin-conjugating enzyme in pediatric acute lymphoblastic leukemia” Cell Growth Differ. 12:427-433 (2001). Rbl2/p130 is known to control progression from G0 into G1 phase of cell cycle, and Rbl2/p130 expression was lower in rats consuming diet containing FO/P compared to CO/C.

However, Rbl2/p130 phosphorylation results in E2F release and activates the cell cycle. FO/P resulted in lower levels of expression of CDKs and GSK2b, which is a reported kinase of Rbl2/p130. Litovchick et al., “Glycogen synthase kinase 3 phosphorylates RBL2/p130 during quiescence” Mol Cell Biol. 24:8970-8980 (2004). Although it is not necessary to understand the mechanism of an invention, it is believed that these shifts in cell cycle regulator expression, potentially leading to lower levels of expression, may reduce the proliferative zone found in the FO/P rats.

These data suggest that FO/P diet could suppress uncontrolled cell proliferation under colon carcinogenesis at the ACF stage in part by modulating expressions of genes that are involved with cell cycle activities. For example, Prkaa1 gene encodes AMP activated protein kinase (AMPK, a cellular energy sensor), and rats fed the FO/P diet had lower levels of expression as compared to rats fed the CO/C diet. Immunohistological assay of cervical cancer tissues, show prkaa1 protein in the basal cell layer, an active proliferation layer. Huang et al., “Semi-quantitative fluorescent PCR analysis identifies PRKAA1 on chromosome 5 as a potential candidate cancer gene of cervical cancer” Gynecol Oncol. 103:219-225 (2006). It has been reported that Prkaa1 may also regulate anaerobic cancer cell energy metabolism as well as prevents cancer cell apoptosis. Kato et al., “Critical roles of AMP-activated protein kinase in constitutive tolerance of cancer cells to nutrient deprivation and tumor formation” Oncogene 21:6082-6090 (2002).

In the Wnt signaling pathway, Ruvbl1 enhances the transcription of Wnt target genes by interacting with β-catenin and was reported to be up-regulated in human colon cancer tissue. Lauscher et al., “Increased pontin expression in human colorectal cancer tissue” Hum Pathol. 38:978-985 (2007). The present data suggests that an FO/P diet may modulate Wnt signaling by downregulating Ruvbl1 expression as compared to CO/C diet.

The localization and expression of 06-methylguanine DNA methyltransferase (MGMT, DNA repair enzyme), has been reported to remove AOM-induced DNA adducts. Hong et al., “Dietary fish oil reduces 06-methylguanine DNA adduct levels in rat colon in part by increasing apoptosis during tumor initiation” Cancer Epidemiol Biomarkers Prev. 9:819-826 (2000). The data herein show that the expression of MGMT in fish oil-fed rats was 4-fold higher than corn oil-fed rats in the position where apoptosis occurred at the initiation stage.

A FO/P diet also showed a 3-fold up-regulation of Pms2_predicted expression as compared to a CO/C diet. Pms2 is believed to be involved in DNA mismatch repair systems and a Pms2 mutation is believed to cause hereditary nonpolyposis colorectal cancer. These observations may suggest that a fish oil/pectin diet may mitigate DNA damage-induced cell cycle interruptions, thereby preventing cell apoptosis. Nicolaides et al., “Mutations of two PMS homologues in hereditary nonpolyposis colon cancer” Nature 371:75-80 (1994). Although it is not necessary to understand the mechanism of an invention, it is believed that a FO/P diet could enhance apoptosis to remove DNA damaged cells as well as suppress cell proliferation.

3. Intermediate Stage

Fourteen (14) weeks after AOM injection colorectal cancer enters the intermediate stage. The data show that a fish oil/pectin diet resulted in lower cell proliferation as compared with a corn oil/cellulose diet. See, FIG. 10. Fecal microarray GO analysis indicated suppression of genes in the cell proliferation pathway by fish oil/pectin led to enrichment within the cell proliferation pathway; GO category: GO:000828; Number of genes: thirty-four (34); p<0.019. For example, some of the down-regulated cell proliferation pathway genes include, but are not limited to: i) beta catenin 1, 88 kDa (BE118486); ii) interleukin 18 (NM_{—}019165); iii) interleukin 1 alpha (NM_{—}017019); and iv) tumor necrosis factor superfamily, member 15 (NM_{—}145765).

4. Tumor Stage

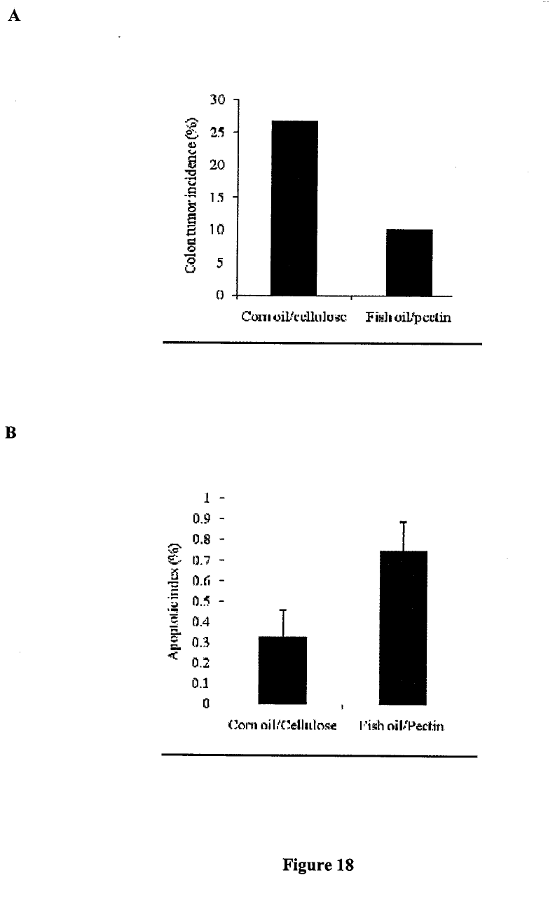

Twenty-eight (28) weeks after AOM injection colorectal cancer enters the tumor stage. Similar to the reduction of early preneoplastic lesion numbers, colon tumor incidence evaluated 31 weeks after the second AOM injection was lower in FO/P fed rats than in CO/C rats. Part of the protection against tumor formation may be attributable to the enhanced apoptotic index in the FO/P rat colons, compared to those from rats consuming CO/C. See, FIG. 18.

At this stage, eighty-one (81) genes were differentially expressed in response to diet, and thirteen (13) biological processes were identified by GO analysis as being enriched at the tumor stage. Of the 13 categories, six were associated with apoptosis. See, Table 6.

TABLE 6
GO terms significantly overrepresented in the genes altered by FO/P diet compared to
CO/C at tumor stage

| | | | Total genes | |
|---|---|---|---|---|
| GO number | GO term (biological process) | Genes in list | in list | P-value |
| GO: 0009628 | Response to abiotic stimulus | 5 | 212 | 0.003182 |
| GO: 0006397 | mRNA Processing | 4 | 152 | 0.009571 |
| GO: 0016071 | mRNA Metabolic Process | 4 | 181 | 0.01531 |
| GO: 0042981 | Regulation of apoptosis | 6 | 525 | 0.016984 |
| GO: 0043067 | Regulation of programmed cell death | 6 | 533 | 0.01802 |
| GO: 0051704 | Multi-organism process | 4 | 224 | 0.026749 |
| GO: 0008284 | Positive regulation of cell proliferation | 4 | 243 | 0.032934 |
| GO: 0043065 | Positive regulation of apoptosis | 4 | 256 | 0.037562 |
| GO: 0043068 | Positive regulation of programmed cell | 4 | 259 | 0.038676 |
| GO: 0051707 | Response to other organism | 3 | 113 | 0.042324 |
| GO: 0006915 | Apoptosis | 6 | 681 | 0.045248 |
| GO: 0012501 | Programmed cell death | 6 | 690 | 0.04743 |
| GO: 0006396 | Rna processing | 4 | 285 | 0.049029 |

Six of the eighty-one (81) annotated genes were within the apoptosis pathway and twenty-two (22) genes were classified to biological categories related to apoptosis, such as signal transduction, metabolism, and cancer. Table 7.

TABLE 7
Differentially expressed genes in response to FO/P diet compared with CO/C at tumor
stage

| | | | | | |
|---|---|---|---|---|---|
| GenBank | | | Relative | | Membrane |
| Accession | Gene symbol | Description | expression¹ | Function | related² |

Apoptosis from GO category

| | | | | | |
|---|---|---|---|---|---|
| NM_181386 | Tmem23 | Transmembrane protein 23 | 0.55 | Anti-apoptosis | Y |
| BI302754, | Id3 | Inhibitor of DNA binding 3 | 2.16 | Pro-apoptosis | |
| NM_013058 | | | | | |
| NM _031054 | Mmp2 | Matrix metallopeptidase 2 | 0.69 | Pro-apoptosis, | Y |
| | | | | tumor invasion | |
| AA999104 | Fem1b | Feminization 1 homolog b (C. elegans) | 0.39 | Pro-apoptosis | |
| | | (predicted) | | | |
| BF542507 | Hipk2 | Homeodomain interacting protein kinase | 0.10 | Pro-apoptosis | |
| | | 2 (predicted) | | | |
| NM_017017 | Hgf | Hepatocyte growth factor | 0.55 | Anti-apoptosis | |

Signal transduction

| | | | | | |
|---|---|---|---|---|---|
| CF113820 | Mtmr4 | Myotubularin related protein 4 (predicted) | 3.88 | TGF beta | |
| | | | | signaling | |
| NM_019268 | Slc8a1 | Solute carrier family 8 (sodium/calcium | 0.41 | Calcium | Y |
| | | exchanger), member 1 | | singaling, Pro- | |
| | | | | apoptosis | |
| AW918423 | Dupd1 | Dual specificity phosphatase and pro | 0.46 | MAPK | |
| | | isomerase domain containing 1 | | signaling | |
| | | (predicted) | | | |
| CB749439 | Ppp1r7 | Protein phosphatase 1, regulatory | 0.61 | MAPK | |
| | | (inhibitor) subunit 7 | | signaling | |
| CB611690 | Mfn1 | Mitofusin 1 | 0.53 | Anti-apoptosis | Y |
| NM_053788 | Stx1a | Syntaxin 1A (brain) | 0.57 | Pro-apoptosis | Y |
| NM_001002835 | Smoc1 | SPARC-related modular calcium binding | 0.54 | Positive | Y |
| | | protein 1 | | regulation of | |
| | | | | cell-substrate | |
| | | | | adhesion | |
| NM_019378 | Snip | SNAP25-interacting protein | 0.70 | Negative | |
| | | | | regulation of | |
| | | | | cell adhesion | |
| NM_053346 | Nrn1 | Neuritin | 0.65 | Hypoxia- | Y |
| | | | | induced genes | |
| NM_130410 | Il23a | Interleukin 23, alpha subunit p19 | 0.56 | Immune | |
| | | | | response | |
| NM_017020 | I16ra | Interleukin 6 receptor, alpha | 0.52 | Immune | Y |
| | | | | response | |
| NM_031089 | Pthr2 | Parathyroid hormone receptor 2 | 0.41 | Cell | Y |
| | | | | proliferation | |

Metabolism

| | | | | | |
|---|---|---|---|---|---|
| CB812866 | Abcg5 | ATP-binding cassette, sub-family G | 0.68 | Metabolism | Y |
| | | (WHITE), member 5 | | | |
| CO562407 | Cyp2s1 | Cytochrome P450, family 2, subfamily s, | 0.62 | Metabolism | Y |
| | | polypeptide 1 | | | |
| NM_080477 | Pfkfb2 | 6-phosphofructo-2-kinase/fructose-2,6- | 0.38 | Metabolism | |
| | | bisphosphatase 2 | | | |
| CB735495 | Crot | Carnitine O-octanoyltransferase | 0.46 | Fatty acid | |
| | | | | metabolic | |
| | | | | process | |
| BF400268 | Dagla | Sn1-specific diacylglycerol lipase alpha | 0.51 | Lipid | Y |
| | | | | metabolic | |
| | | | | process | |
| NM_053350 | Agps | Alkylglycerone phosphate synthase | 0.57 | Lipid | |
| | | | | biosynthesis | |
| BF544565 | Phgdhl1 | Phosphoglycerate dehydrogenase like 1 | 0.17 | Glycolysis | |

Tumor

| | | | | | |
|---|---|---|---|---|---|
| NM_001009605 | Brms1 | Breast cancer metastasis-suppressor 1 | 2.60 | Cancer | |
| | | | | metastasis | |
| BI299377 | Rbbp6 | Retinoblastoma binding protein 6 | 2.03 | Tumor | |
| | | | | suppressor | |
| NM_020302 | Adam3 | A disintegrin and metalloprotease domain | 0.58 | Tumor | Y |
| | | 3 (cyritestin) | | invasion | |

¹Relative expression was calculated by FO/P divided by CO/C expression level. Less than 1.00 indicates that the relative gene expression level was lower in rats consuming FO/P diet compared to CO/C. All genes have p-value less than 0.05, indicating that genes were differentially expressed by diet.
²Y indicated that that gene is included in the membrane category (GO data, cellular component).

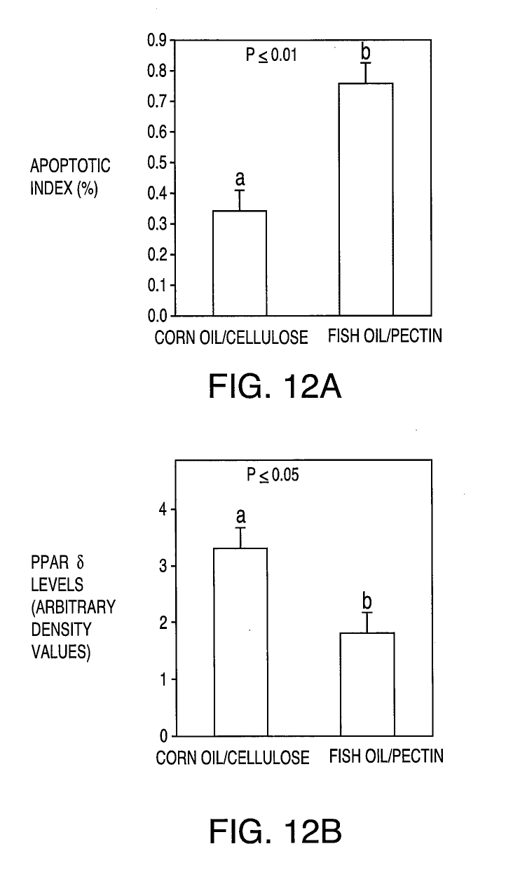

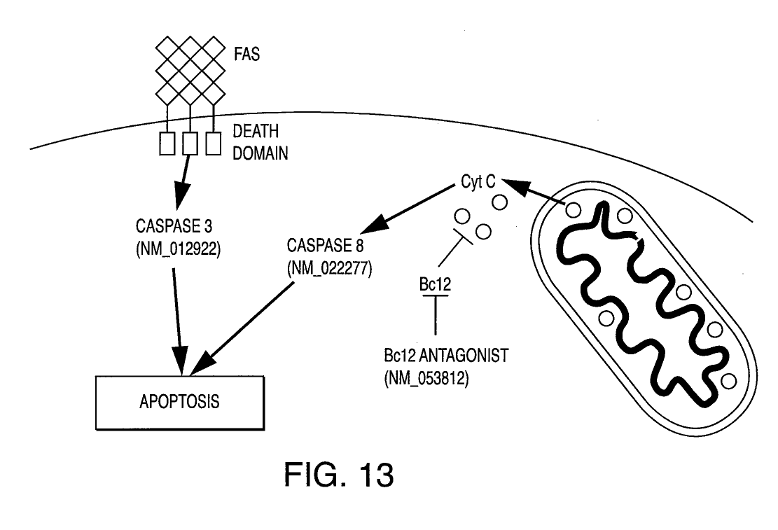

The data show that a fish oil/pectin diet resulted in a higher apoptotic index and PPAR δ receptor level as compared with a corn oil/cellulose diet. See, FIG. 12A and FIG. 12B, respectively. Fecal microarray GO analysis indicated upregulated enrichments in apoptosis pathway genes: GO category: GO:0006915; Number of Genes: thirty-four (34); p<0.006. For example, some of the upregulated apoptotic pathway genes include, but are not limited to: i) caspase 3 (NM_{—}012922); ii) caspase 8 (NM_{—}022277); or iii) bcl2 antagonist (NM_{—}053812). Fecal microarray GO analysis indicated downregulated enrichments in peroxisome proliferator-activated receptor signaling pathway genes (KEGG pathway): GO category: mo03320; Number of Genes: seven (7); p<0.056. One possible pathway for their concerted interaction is provided. See, FIG. 13.

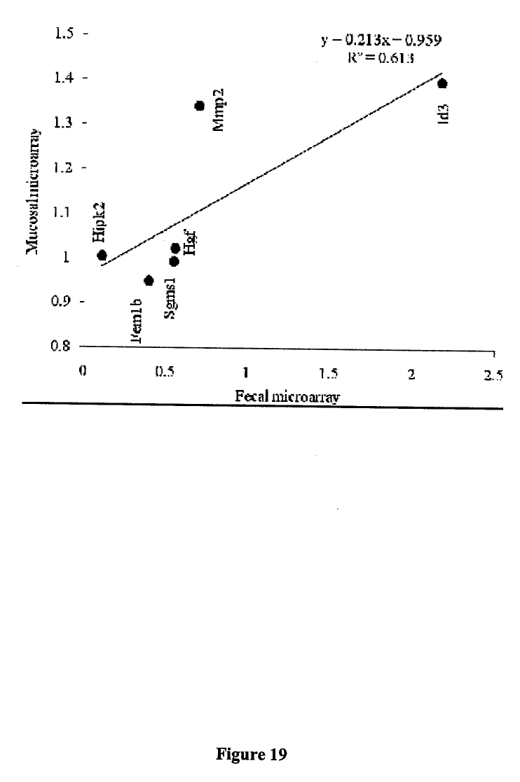

Because apoptosis may be a physiological process affected by diet at all tumor development stages, the expression of six genes within the apoptosis GO category was compared between fecal and mucosal arrays. A regression analysis between fecal and mucosal microarray data demonstrated a reasonable degree of similarity in the pattern of expression detected in both samples (R2=0.613). See, FIG. 19. Data are presented as the ratio of the expression level in FO/P-fed rats to that of CO/C-fed rats.

Overall, these data show that colon tumor incidence was lower in FO/P fed rats, compared with CO/C rats. Elevated apoptosis was first observed at the initiation and ACF stages, continued through the tumor stage, with rats consuming the FO/P diet having a larger apoptotic index compared to the CO/C rats. Although it is not necessary to understand the mechanism of an invention, it is believed that a lower number of colon tumor incidence may, in part, be due to the induction of apoptosis that occurs with the FO/P diet. The induction of apoptosis in the FO/P rats occurred in concert with the modulation of expression of several genes involved in apoptosis. For example, Mmp2 expression is increased in colon tumors and has been implicated in colon cancer invasion. Tutton et al., “Use of plasma MMP-2 and MMP-9 levels as a surrogate for tumour expression in colorectal cancer patients” Int J Cancer 107:541-550 (2003). In contrast, tissues from the FO/P rats had lower Mmp2 expression as compared to a CO/C rats.

Additionally, Id3, an apoptosis inducer, was observed to have a higher expression in FO/P rats as compared to CO/C, whereas Tmem23 and Hgf, both apoptosis inhibitors, were lower in FO/P rats compared to CO/C. Norton et al., “Coupling of cell growth control and apoptosis functions of Id proteins” Mol Cell Biol. 18:2371-2381 (1998); Xu et al., “High tolerance to apoptotic stimuli induced by serum depletion and ceramide in side-population cells: high expression of CD55 as a novel character for side-population” Exp Cell Res. 313:1877-1885 (2007); and Kitamura et al., “Met/HGF receptor modulates bcl-w expression and inhibits apoptosis in human colorectal cancers” Br J Cancer 83:668-673 (2000). In one embodiment, the present invention contemplates a method showing a pattern of differences in gene expression wherein a FO/P diet is more effective in inducing apoptosis than a CO/C diet. Although it is not necessary to understand the mechanism of an invention, it is believed that these data are consistent with previous reports suggesting that FO/P enhances colonocyte apoptosis through suppression of PPARδ/PGE2 and elevation of PGE3. Vanamala et al., “Dietary fish oil and pectin enhance colonocyte apoptosis in part through suppression of PPARdelta/PGE2 and elevation of PGE3” Carcinogenesis 29:790-796 (2008).

The data presented herein demonstrate expression of several genes involved in signal transduction were down-regulated in FO/P fed rats as compared to a CO/C diet. These data suggest that a FO/P diet could attenuate downstream signaling pathways. For example, Mfn1, encoding a transmembrane GTPase, may mediate mitochondrial fusion and increase the cell resistance to death stimuli. Zhang et al., “New insights into mitochondrial fusion” FEBS Lett. 581:2168-2173 (2007). On the other hand, another signal transduction gene, Mtmr4, was up-regulated by a fish oil/pectin diet, and is believed to prevent overactivation of TGF β signaling. Yu et al., “MTMR4 attenuates TGF {beta} signaling by dephosphorylating R-Smads in endosomes” J Biol Chem. E-pub, (Jan. 8, 2010).

Fish oil has been reported to enhance the generation of reactive oxygen species and possibly induce apoptosis by incorporation into mitochondrial membrane phospholipid. Chapkin et al., “Dietary n-3 PUFA alter colonocyte mitochondrial membrane composition and function” Lipids 37:193-199 (2002). Although it is not necessary to understand the mechanism of an invention, it is believed that at least thirteen (13) genes in the membrane category (GO data, cellular components) were identified as differentially expressed genes at the tumor stage. It is further believed that fish oil n-3 PUFAs are incorporated into cell membranes and/or mitochondria membranes. Hong et al., “Fish oil increases mitochondrial phospholipid unsaturation, upregulating reactive oxygen species and apoptosis in rat colonocytes” Carcinogenesis 23:1919-1925 (2002).

In addition to the present observations that tumor cells exhibit altered metabolic phenotypes it has been reported that glycolytic genes were overexpressed in various cancer types. Altenberg et al., “Genes of glycolysis are ubiquitously overexpressed in 24 cancer classes” Genomics 84:1014-1020 (2004). A FO/P diet as compared to a CO/C diet suppressed metabolism related gene expression, including the Pfkfb2 gene that encodes a glycolytic regulatory enzyme. Similarly, a FO/P diet suppressed Cyp2s1 expression as compared to a CO/C diet. Cyp2s1 encodes a cytochrome P450 superfamily enzyme and plays a role in the oxidative metabolism of xenobiotics such as carcinogens. It has been reported that Cyp2s1 expression may be significantly higher in primary colon cancer than a normal colon. Kumarakulasingham et al., “Cytochrome p450 profile of colorectal cancer: identification of markers of prognosis” Clin Cancer Res. 11:3758-3765 (2005).

In general, the present data show that tumor related genes were beneficially controlled by FO/P consumption compared to CO/C. For example, Brms1, a tumor suppressor, was up-regulated following a fish oil/pectin diet. In contrast, Adam 3, an indicator of tumor invasion, was down-regulated in FO/P rats compared to a CO/C diet. Zhang et al., “Suppression of human ovarian carcinoma metastasis by the metastasis-suppressor gene, BRMS1” Int J Gynecol Cancer 522-531 (2006); and Arribas et al., “ADAMs, cell migration and cancer” Cancer Metastasis Rev. 25:57-68 (2006), respectively.

At the tumor stage, the present data show that a FO/P diet contributed to a 2-fold increase in Rbbp6 expression. Rbbp6 is also known as P2P-R, whose overexpression has been reported to result in mitotic arrest at prometaphase and mitotic apoptosis. Gao et al., “P2P-R protein overexpression restricts mitotic progression at prometaphase and promotes mitotic apoptosis” J Cell Physiol. 193:199-207 (2002). Consequently, these changes in gene expression by FO/P might lead chemoprotective effect against colon carcinogenesis with enhanced apoptosis as the central mechanism at tumor stage.

These data demonstrate that disease progression and dietary protection can be monitored using a non-invasive technique. Specifically, the data indicate that fish oil/pectin suppresses colon carcinogenesis through stage-dependent changes in gene expression: i) at the ACF and tumor stages, colon carcinogenesis in suppressed by induction of apoptosis; ii) at the intermediate stage, colon carcinogenesis is suppressed by cell proliferation.

In one embodiment, the present invention contemplates a method for tracking changes in gene expression over time using a non-invasive technique. In one embodiment, the method monitors disease development. In one embodiment, the method identifies biological stages wherein dietary intervention modulates disease development. In one embodiment, the method identifies a fish oil/pectin diet suppresses colon cancer initiation, growth, and/or tumor development. In one embodiment, the fish oil/pectin diet up-regulates genes regulating apoptosis pathways.

### VI. Dietary Protection of Radiation Induced Cell Proliferation

In one embodiment, the present invention contemplates a method providing a diet high in fish oil and pectin. In one embodiment, the fish oil/pectin diet is more effective than a corn oil/cellulose diet in mitigating Wnt/β-catenin signaling pathway activation following radiation exposure (i.e., for example, 1 Gy, 1 GeV/nucleon Fe ions at Brookhaven National Laboratory). In one embodiment, the method further comprises measuring a time course of gene expression profiles from fecal RNA. Loktionov A., “Cell exfoliation in the human colon: myth, reality and implications for colorectal cancer screening” Int J Cancer 120:2281-2289 (2007). In one embodiment, the fecal RNA comprises poly (A)+ RNA extracted from fecal colonocytes. In one embodiment, the method further comprises identifying the poly (A)+ RNA on a microarray. Although it is not necessary to understand the mechanism of an invention, it is believed that the identified RNA fragments can be subjected to a biochemical pathway analysis to determine which gene pathways and/or gene targets were affected by the interaction of diet and radiation exposure. The data presented herein suggest that radiation exposure may promote colon carcinogenesis that can be suppressed by a fish oil/pectin diet.

In one embodiment, the present invention contemplates a method comprising providing radiation exposure generating a first gene expression pattern, thereby promoting colon carcinogenesis. In one embodiment, the method further comprises a fish oil/pectin diet generating a second gene expression pattern, thereby suppressing colon carcinogenesis.

The data presented herein identifies differences between carcinogenic protective and carcinogenic promotive diets as measured using gene expression patterns from exfoliated rat colonocytes exposed to radiation (+/−1 Gy, 1 GeV/nucleon Fe ions) and a chemical carcinogen (AOM, 2×, 15 mg/kg BW). This is the first time that a protective effect of fish oil/pectin combination on colon carcinogenesis has been reported to partially counteract the negative effects of radiation on colon health. Although it is not necessary to understand the mechanism of an invention, it is believed that radiation affected expression of genes within the Wnt/β-catenin signaling pathway, which would be positive regulators of the pathway and promote colon carcinogenesis, but the fish oil/pectin diet reduced expression of genes related to cell adhesion and receptor activity, which would be negative regulators of Wnt/β-catenin signaling pathway and suppress colon carcinogenesis.

A. Preliminary Observations

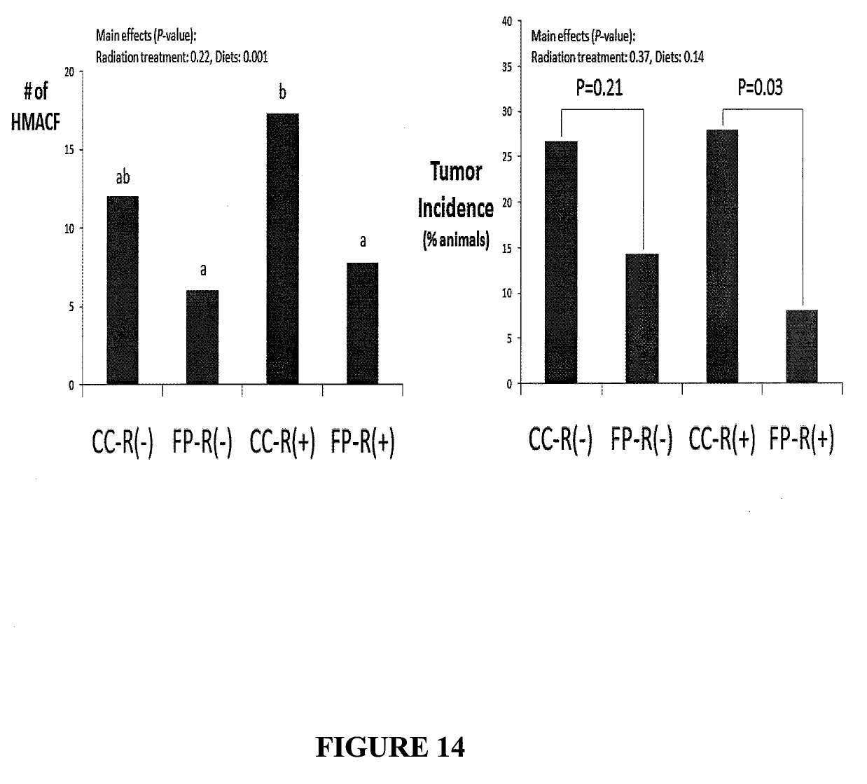

Preliminary data identified changes during the tumor promotion stage, wherein phenotypic observations indicated that radiation treatment increased the number of HMACF (i.e., for example, abberant crypt foci (ACF) with four or more aberrant crypts per foci). FIG. 14A. It was believed that these observations were a result of increasing cell proliferation (p<0.05), and/or decreasing apoptosis (p=0.034). A fish oil/pectin diet reduced HMACF number (p<0.05) compared to corn oil/cellulose diet. Further, the data suggested that pectin intake induced apoptosis, as compared to cellulose intake. Data collected during the tumor stage, showed that a fish oil/pectin diet had improved anticancer effects against radiation-enhanced colon carcinogenesis as compared a corn oil/cellulose diet. FIG. 14B. The data suggest that intra-diet tumor incidence was equivalent between the respective irradiated and non-irradiated groups, but the considerations about skin cancer incidence in irradiated groups may support the radiation enhanced cancer.

Based on these preliminary results, a detailed examination of how radiation exposure and diet intake altered expression of genes involved in carcinogenesis, proliferation and apoptosis.

B. Interactive Effects of Diet and Radiation on Carcinogenesis

Of the 35,129 gene probes in the CodeLink® Rat Whole Genome Bioarray, 9,037 reliable gene expressions remained after data normalization from fecal poly A (+) RNA generated from 5,904 known genes. A DAVID analysis of the data presented herein demonstrate that eighty nine (89) genes were differentially expressed due to radiation, two hundred and fifty seven (257) genes were differentially expressed due to diet, and sixty four (64) genes were differentially expressed due to the interaction of radiation and diet. (significance adjusted p-value<0.05).

1. Radiation Main Effects

A functional analysis of the eighty nine (89) differentially expressed genes that were correlated only with radiation exposure identified at least twenty-two (22) GO biological processes that were overrepresented including, but not limited to, post translational protein modification, cyclic nucleotide mediated signaling, MAPKKK cascade(s), and one GO cellular component (cytoplasm; p=0.0365; n=26). See, Table 8.

TABLE 8
Functional categories with significant enrichment of genes differentially expressed by
the radiation treatment.

| | | | | |
|---|---|---|---|---|
| | | | Irradiated/ | |
| Symbol | Gene name | GenBank ID | Non-irradiated | p-value |

GO Biological Processes
Post-translational protein modification (p = 0.0031)

| | | | | |
|---|---|---|---|---|
| Vrk3 | VACCINIA RELATED KINASE 3 | BE329415 | 2.4521 | 0.0069 |
| Crebbp | CREB BINDING PROTEIN | AI137114 | 2.0111 | 0.0099 |
| Ube2q | UBIQUITIN-CONJUGATING ENZYME E2Q (PUTATIVE) | AA800501 | 0.5155 | 0.0103 |
| Dusp5 | DUAL SPECIFICITY PHOSPHATASE 5 | NM_133578 | 0.6639 | 0.0134 |
| Ube2g1 | UBIQUITIN-CONJUGATING ENZYME E2G 1 (UBC7 | BM391675 | 1.8712 | 0.0278 |
| | HOMOLOG, C. ELEGANS) | | | |
| Met | MET PROTO-ONCOGENE | NM_031517 | 0.4374 | 0.0283 |
| F2r | COAGULATION FACTOR II (THROMBIN) RECEPTOR | NM_012950 | 1.3482 | 0.0284 |
| Map3k7 | MITOGEN ACTIVATED PROTEIN KINASE KINASE | CA503814 | 0.6109 | 0.0300 |
| | KINASE 7 | | | |
| Ppp3ca | PROTEIN PHOSPHATASE 3, CATALYTIC SUBUNIT, | BQ210125 | 0.4740 | 0.0312 |
| | ALPHA ISOFORM | | | |
| Pctk3 | PCTAIRE-MOTIF PROTEIN KINASE 3 | AA956305 | 0.6029 | 0.0363 |
| Arih1 | ARIADNE UBIQUITIN-CONJUGATING ENZYME E2 | AI231517 | 0.5987 | 0.0376 |
| | BINDING PROTEIN HOMOLOG 1 (DROSOPHILA) | | | |
| Gucy2d | GUANYLATE CYCLASE 2D | NM_130737 | 1.3708 | 0.0387 |
| Ephb1 | EPH RECEPTOR B1 | M59814 | 0.7081 | 0.0393 |
| Bre | BRAIN AND REPRODUCTIVE ORGAN-EXPRESSED | AA818011 | 3.0105 | 0.0427 |
| | PROTEIN | | | |

Cyclic-nucleotide-mediated signaling (p = 0.0309)

| | | | | |
|---|---|---|---|---|
| Gnat1 | GUANINE NUCLEOTIDE BINDING PROTEIN, ALPHA | BG371798 | 1.3813 | 0.0017 |
| | TRANSDUCING 1 | | | |
| eIF4Ebp2 | EUKARYOTIC TRANSLATION INITIATION FACTOR 4E | BF559340 | 1.8050 | 0.0035 |
| | BINDING PROTEIN 2 | | | |
| Adcy7 | ADENYLATE CYCLASE 7 | CB759352 | 1.7814 | 0.0062 |
| Htr2c | 5-HYDROXYTRYPTAMINE (SEROTONIN) RECEPTOR 2C | NM_012765 | 0.7260 | 0.0360 |

Metal ion transport (p = 0.0431)

| | | | | |
|---|---|---|---|---|
| Ndufa9 | NADH DEHYDROGENASE (UBIQUINONE) 1 ALPHA | AI102086 | 0.7027 | 0.0263 |
| | SUBCOMPLEX, 9 | | | |
| F2r | COAGULATION FACTOR II (THROMBIN) RECEPTOR | NM_012950 | 1.3482 | 0.0284 |
| Ppp3ca | PROTEIN PHOSPHATASE 3, CATALYTIC SUBUNIT, | BQ210125 | 0.4740 | 0.0312 |
| | ALPHA ISOFORM | | | |
| Accn3 | AMILORIDE-SENSITIVE CATION CHANNEL 3 | NM_173135 | 0.4281 | 0.0336 |
| Kcnh7 | POTASSIUM VOLTAGE-GATED CHANNEL, SUBFAMILY | NM_131912 | 0.6736 | 0.0350 |
| | H (EAG-RELATED), MEMBER 7 | | | |
| Slc39a6 | SOLUTE CARRIER FAMILY 39 (METAL ION | BF397041 | 0.5285 | 0.0376 |
| | TRANSPORTER), MEMBER 6 | | | |

MAPKKK cascade (p = 0.0456)

| | | | | |
|---|---|---|---|---|
| Rgs3 | REGULATOR OF G-PROTEIN SIGNALLING 3 | BF418764 | 1.6854 | 0.0237 |
| Met | MET PROTO-ONCOGENE | NM_031517 | 0.4374 | 0.0283 |
| F2r | COAGULATION FACTOR II (THROMBIN) RECEPTOR | NM_012950 | 1.3482 | 0.0284 |
| Map3k7 | MITOGEN ACTIVATED PROTEIN KINASE KINASE | CA503814 | 0.6109 | 0.0300 |
| | KINASE 7 | | | |

Orphan genes

| | | | | |
|---|---|---|---|---|
| Fbxw11 | F-BOX AND WD-40 DOMAIN PROTEIN 11 | BF523211 | 0.7008 | 0.0073 |
| Hbp1 | IGH MOBILITY GROUP BOX TRANSCRIPTION FACTOR 1 | NM_013221 | 0.3531 | 0.0103 |
| Hdac4 | HISTONE DEACETYLASE 4 | BM390113 | 3.8718 | 0.0262 |
| Cnbp1 | CELLULAR NUCLEIC ACID BINDING PROTEIN 1 | NM_022598 | 1.7925 | 0.0475 |

In general, these data suggest that radiation exposure altered the gene expression profile of genes associated with the cell cytoplasm.

a. Post-Translational Protein Modification Genes

Post-translational protein modifications, which relates to chemical modifications including, but not limited to, acylation, methylation, and/or phosphorylation of a protein after its translation was observed to be the biological process with the lowest p value (i.e., for example, the most correlated by statistical significance). The data show that radiation treatment caused up-regulation of genes including, but not limited to, Bre, Crebb, F2r, Gucy2d, Ube2g, and Vrk3, and down-regulation of genes including, but not limited to, Arih1, Dusp5, Ephb1, Map3k7, Met, Pctk3, Ppp3ca and Ube2q. Six of these genes, Crebb, Ephb1, F2r, Map3k7, Met, and Ppp3ca, may either, directly or indirectly, play a role in modulating post-translational modifications of the components of the Wnt/β-catenin signaling pathway.

One of these post-translational modification genes is Coagulation factor II (thrombin) receptor (F2r). F2r is believed to encode a family of G protein coupled receptors, known as thrombin receptor PAR1, and may play a role in cell proliferation and carcinogenesis (i.e., for example, colon cancer). In relation to the Wnt/β-catenin signaling pathway, F2r may facilitate nuclear localization of β-catenin through Dsh phosphorylation. Yin et al., “Mammary gland tissue targeted overexpression of human protease-activated receptor 1 reveals a novel link to beta-catenin stabilization” Cancer Res 66:5224-5233 (2006).

A third representative post-translational modification gene is CREB binding protein (Crebbp). Crebbp is believed to be composed of transcriptional coactivators with p300, and interact with transcription factors thereby increasing target gene expression. Although it is not necessary to understand the mechanism of an invention, it is believed that Crebbp may acetylate β-catenin to stabilize it, induce its nuclear translocation, and increase its transcriptional effects in β-catenin-driven diseases (i.e., for example, colon cancer). Ge et al., “PCAF acetylates {bet}-catenin and improves its stability” Mol Biol Cell 20:419-427 (2009). It is further believed that radiation induction of a Crebbg complex by radiation could play a role in radiation-induced Wnt signaling activation.

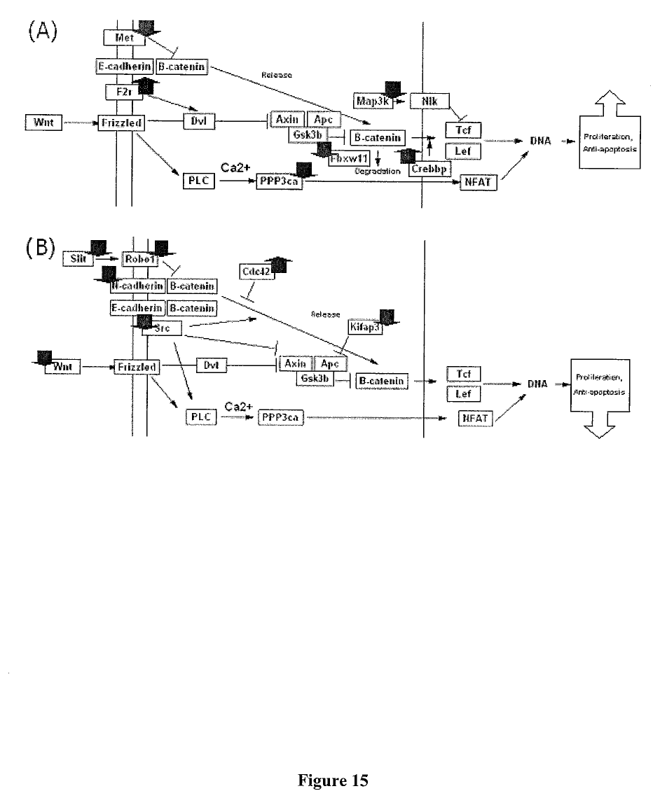

A fourth representative post-translational modification gene is mitogen-activated protein kinase kinase kinase 7 (Map3k7), also known as TGF-β activated kinase 1 (Tak1). Map3k7 is believed to activate a mitogen-activated protein kinase pathway by activating NF-kB and JNK/p38 kinase pathways. Wang et al., “TAK1 is a ubiquitin-dependent kinase of MKK and IKK” Nature 412:346-351 (2001). Nonetheless, it has been reported that Map3k7 stimulation activates Nemo-like kinase (Nlk), resulting in Tcf phosphorylation antagonism of Wnt/β-catenin signaling. Ishitani et al., “The TAK1-NLK mitogen-activated protein kinase cascade functions in the Wnt-5a/Ca(2+) pathway to antagonize Wnt/beta-catenin signaling” Mol Cell Biol 23:131-139 (2003). The data described herein indicates that reductions in post-translational gene expression by radiation treatment might contribute to the activation of the Wnt/β-catenin signaling pathway. FIG. 15.

Intestinal adenomas are believed to be characterized by high expression of Wnt target genes, including, but not limited to, EPH receptor B1 (EphB1). However, as adenomas become aggressive, the expression of EphB receptors is silenced despite the persistence of Wnt pathway mutations, and may promote the formation of aggressive colorectal tumors. The data presented herein show that Ephb1 expression was suppressed by radiation treatment, and as such, might affect the formation of aggressive colorectal tumors. Batlle et al., “Beta-catenin and TCF mediate cell positioning in the intestinal epithelium by controlling the expression of EphB/ephrinB” Cell 111:251-263 (2002).

A mesenchymal-epithelial transition (Met) protein, is believed to be a tyrosine kinase receptor and forms a complex with β-catenin. Although it is not necessary to understand the mechanism of an invention, it is believed that the Met/β-catenin complex may play a role in carcinogenesis by undergoing dissociation on stimulation by hepatocyte growth factor thereby activating an oncogenic pathway like the nuclear translocation of β-catenin. Monga et al., “Hepatocyte growth factor induces Wnt-independent nuclear translocation of beta-catenin after Met-beta-catenin dissociation in hepatocytes” Cancer Res 62:2064-2071 (2002). The present data show that radiation treatment reduced Met expression, which may cause the accumulation of free β-catenin in the cytoplasm or nucleus.

Protein phosphatase 3 catalytic subunit, alpha isoform (Ppp3ca), is believed to be a component of calcineurin. Calcineurin is a calcium regulated enzyme that participates in the signaling cascades involved in the Wnt/Ca²⁺ pathway. While the present data suggests that Ppp3ca is unaffected by radiation exposure, it has been reported that Ppp3ca expression is elevated in human colorectal adenocarcinomas. Lakshmikuttyamma et al., “Increased expression of calcineurin in human colorectal adenocarcinomas” J Cell Biochem 95:731-739 (2005). One explanation may be because the Wnt/Ca²⁺ pathway is known to interfere with the Wnt/β-catenin pathway, so down-regulation of the Wnt/Ca²⁺ pathway might increase Wnt/β-catenin pathway activity.

Interestingly, three post-translational modification genes that may be related to Ubiquitin mediated proteolysis were differentially effected by radiation exposure. Ubiquitin-conjugating enzyme E2G1 (Ube2g1) showed a significant increase in expression, while Ubiquitin-conjugating enzyme E2Q (Ube2q) and Ariadne ubiquitin-conjugating enzyme E2 binding protein homolog 1 (Arih1) showed significant decreases in expression. Interestingly, the orphan “F-box and WD repeat domain containing 11 gene” (Fbxw11) may constitute one of the ubiquitin protein ligase complexes, whose function is believed to specifically interact with phosphorylated β-catenin, thereby targeting phosphorylated β-catenins for proteasome-dependent ubiquitination. Fuchs et al., “HOS, a human homolog of Slimb, forms an SCF complex with Skp1 and Cullin1 and targets the phosphorylation-dependent degradation of IkappaB and beta-catenin” Oncogene 18:2039-2046 (1999). The data indicate that radiation exposure reduced the expression of Arih1, Ube2q and Fbxw11, and consequently β-catenin ubiquitination may also be concomitantly reduced, thereby increasing β-catenin dependent transcription activity.

Brain and reproductive organ-expressed protein (Bre) attenuates death receptor-initiated apoptosis in cancer cells. Chan et al., “BRE is an antiapoptotic protein in vivo and overexpressed in human hepatocellular carcinoma” Oncogene 27:1208-1217 (2008). The data shown herein indicates that Bre over-expression by radiation treatment might promote tumorigenesis.

Dual specificity phosphatase 5 (Dusp5) has been reported to be a direct transcriptional target of tumor suppressor p53 in colon cancer cells, and may be involved in p53-dependent suppression of cell growth in deactivating the MAPK pathway. Ueda et al., “Dual-specificity phosphatase 5 (DUSP5) as a direct transcriptional target of tumor suppressor p53” Oncogene 22:5586-5591 (2003). The data herein suggest that radiation exposure down-regulates Dusp5 and might be related to colon carcinogenesis.

b. Orphan Genes

The data described in Table 3 identified several genes that were differentially affected by radiation that did not associate with other genes in the same family. These gene expression patterns suggest that radiation may enhance colon cancer via increasing cell proliferation.

Histone deacetylase 4 (Hdac4) has been suggested to induce colon cancer cell growth via repression of p21 promoter activity. Hdac4 over-expression by radiation treatment can potentially suppress expression of p21, and induce colon carcinogenesis. Wilson et al., “HDAC4 promotes growth of colon cancer cells via repression of p21” Mol Biol Cell 19:4062-4075 (2008).

HMG-box transcription factor 1 (Hbp1) is a transcriptional repressor that inhibits Wnt/b-catenin target gene expression such as cyclin D1. Sampson et al., “Negative regulation of the Wnt-beta-catenin pathway by the transcriptional repressor HBP1” EMBO J 20:4500-4511 (2001). The down-regulation of Hbp1 by radiation exposure might activate the transcription of Wnt/β-catenin target gene.

The transcription factor cellular nucleic acid-binding protein 1 (Cnbp1) has been reported to stimulate cell proliferation and increase c-myc promoter activity. Shimizu et al., “Molecular cloning, developmental expression, promoter analysis and functional characterization of the mouse CNBP gene” Gene 307:51-62 (2003). The data herein shows an overexpression of Cnbp1 following radiation exposure.

2. Dietary Main Effects

A functional analysis of the two hundred and fifty seven (257) differentially expressed genes that were correlated only with diet consumption identified at least six (6) GO biological processes including, but not limited to, cell adhesion (p=0.043, n=13), at least four GO cellular components, including, but not limited to, the cell membrane (p=0.0419, n=80), and at least eight GO molecular functions, including, but not limited to, receptor activity (p=0.0352; n=24). See, Table 9.

TABLE 9
Functional categories with significant enrichment of genes differentially expressed by
fish oil/pectin diet intake.

| | | | | |
|---|---|---|---|---|
| | | | | p- |
| Symbol | GenBank ID | Gene name | FF/CC | value |

GO Biological
Processes
Cell adhesion (p = 0.0432)

| | | | | |
|---|---|---|---|---|
| Kifap3 | KINESIN-ASSOCIATED PROTEIN 3 | BF401730 | 0.4347 | 0.0001 |
| Cd36 | CD36 ANTIGEN | NM_031561 | 0.6207 | 0.0014 |
| Robo1 | ROUNDABOUT HOMOLOG 1 (DROSOPHILA) | CA504356 | 0.6602 | 0.0016 |
| Cd97 | CD97 ANTIGEN | BF521754 | 0.3567 | 0.0027 |
| Cd47 | CD47 ANTIGEN (RH-RELATED ANTIGEN, | BF392614 | 0.6638 | 0.0056 |
| | INTEGRIN-ASSOCIATED SIGNAL TRANSDUCER) | | | |
| Nell2 | NEL-LIKE 2 HOMOLOG (CHICKEN) | BF415816 | 0.5860 | 0.0056 |
| Col5a3 | COLLAGEN, TYPE V, ALPHA 3 | NM_021760 | 0.4061 | 0.0196 |
| Tspan1 | TETRASPAN 1 | BE349699 | 0.5392 | 0.0225 |
| Cdc42 | CELL DIVISION CYCLE 42 HOMOLOG (S. CEREVISIAE) | H31319 | 2.0293 | 0.0335 |
| Cdh2 | CADHERIN 2 | NM_031333 | 0.6685 | 0.0384 |
| Lpp | LIPOMA-PREFERRED-PARTNER GENE | CF108533 | 1.5779 | 0.0391 |
| Src | ROUS SARCOMA ONCOGENE | NM_031977 | 0.5490 | 0.0443 |
| Aoc3 | AMINE OXIDASE, COPPER CONTAINING 3 | CA334759 | 1.2969 | 0.0474 |

GO Molecular
functions
Ion transmembrane transporter activity (p = 0.0207)

| | | | | |
|---|---|---|---|---|
| Fxyd5 | FXYD DOMAIN-CONTAINING ION TRANSPORT | NM_021909 | 0.3590 | 0.0000 |
| | REGULATOR 5 | | | |
| Slc6a11 | SOLUTE CARRIER FAMILY 6 | NM_024372 | 0.5799 | 0.0000 |
| | (NEUROTRANSMITTER TRANSPORTER, GABA), | | | |
| | MEMBER 11 | | | |
| Grik5 | GLUTAMATE RECEPTOR, IONOTROPIC, KAINATE 5 | NM_031508 | 0.6731 | 0.0008 |
| Kcnq2 | POTASSIUM VOLTAGE-GATED CHANNEL, | BE110018 | 0.5606 | 0.0044 |
| | SUBFAMILY Q, MEMBER 2 | | | |
| Gabrr2 | GAMMA-AMINOBUTYRIC ACID A RECEPTOR, | NM_017292 | 0.7802 | 0.0064 |
| | RHO 2 | | | |
| Sfxn5 | SIDEROFLEXIN 5 | BF403867 | 0.6207 | 0.0157 |
| Slc8a1 | SOLUTE CARRIER FAMILY 8 (SODIUM/CALCIUM | NM_019268 | 0.7130 | 0.0189 |
| | EXCHANGER), MEMBER 1 | | | |
| Slc39a4 | SOLUTE CARRIER FAMILY 39 (ZINC | BQ200090 | 0.4656 | 0.0192 |
| | TRANSPORTER), MEMBER 4 | | | |
| Atp5g2 | ATP SYNTHASE, H+ TRANSPORTING, | NM_133556 | 2.0308 | 0.0221 |
| | MITOCHONDRIAL F0 COMPLEX, SUBUNIT C | | | |
| | (SUBUNIT 9), ISOFORM 2 | | | |
| Kctd14 | POTASSIUM CHANNEL TETRAMERISATION | BI282914 | 0.3453 | 0.0262 |
| | DOMAIN CONTAINING 14 | | | |
| Atp2a1 | ATPASE, CA++ TRANSPORTING, CARDIAC | NM_058213 | 0.6079 | 0.0275 |
| | MUSCLE, FAST TWITCH 1 | | | |
| Kcnab1 | POTASSIUM VOLTAGE-GATED CHANNEL, | BF396103 | 0.4763 | 0.0279 |
| | SHAKER-RELATED SUBFAMILY, BETA MEMBER 1 | | | |
| Slc39a11 | SOLUTE CARRIER FAMILY 39 (METAL ION | BF553125 | 0.6840 | 0.0285 |
| | TRANSPORTER), MEMBER 11 | | | |
| Slc6a18 | SOLUTE CARRIER FAMILY 6 | NM_017163 | 0.5575 | 0.0387 |
| | (NEUROTRANSMITTER TRANSPORTER), MEMBER | | | |
| | 18 | | | |
| Atp7b | ATPASE, CU++ TRANSPORTING, BETA | NM_012511 | 0.5483 | 0.0396 |
| | POLYPEPTIDE | | | |

Receptor activity (p = 0.0352)

| | | | | |
|---|---|---|---|---|
| Thrap1 | THYROID HORMONE RECEPTOR ASSOCIATED | AA999176 | 0.2912 | 0.0000 |
| | PROTEIN 1 | | | |
| Pthr2 | PARATHYROID HORMONE RECEPTOR 2 | NM_031089 | 0.4911 | 0.0001 |
| Cd79b | CD79B ANTIGEN | CA503960 | 0.5123 | 0.0004 |
| Grik5 | GLUTAMATE RECEPTOR, IONOTROPIC, KAINATE 5 | NM_031508 | 0.6731 | 0.0008 |
| Kiss1r | G PROTEIN-COUPLED RECEPTOR 54 | NM_023992 | 0.5165 | 0.0009 |
| Cd36 | CD36 ANTIGEN | NM_031561 | 0.6207 | 0.0014 |
| Robo1 | ROUNDABOUT HOMOLOG 1 (DROSOPHILA) | CA504356 | 0.6602 | 0.0016 |
| Il12rb2 | INTERLEUKIN 12 RECEPTOR, BETA 2 | BF563136 | 0.7086 | 0.0020 |
| Cd97 | CD97 ANTIGEN | BF521754 | 0.3567 | 0.0027 |
| Gfra3 | GLIAL CELL LINE DERIVED NEUROTROPHIC | AA925330 | 0.6921 | 0.0028 |
| | FACTOR FAMILY RECEPTOR ALPHA 3 | | | |
| Gabrr2 | GAMMA-AMINOBUTYRIC ACID A RECEPTOR, | NM_017292 | 0.7802 | 0.0064 |
| | RHO 2 | | | |
| Olr1654 | OLFACTORY RECEPTOR 1654 | NM_021860 | 0.6830 | 0.0072 |
| Rxra | RETINOID X RECEPTOR ALPHA | CB707601 | 0.4153 | 0.0078 |
| Glp1r | GLUCAGON-LIKE PEPTIDE 1 RECEPTOR | NM_012728 | 0.5101 | 0.0085 |
| Sectm1a | SEC63-LIKE (S. CEREVISIAE) | AI105283 | 0.5112 | 0.0105 |
| Grm7 | GLUTAMATE RECEPTOR, METABOTROPIC 7 | NM_031040 | 0.6190 | 0.0116 |
| Htr2c | 5-HYDROXYTRYPTAMINE (SEROTONIN) | NM_012765 | 0.6925 | 0.0151 |
| | RECEPTOR 2C | | | |
| Ogfrl1 | OPIOID GROWTH FACTOR RECEPTOR-LIKE 1 | AI105154 | 0.6471 | 0.0156 |
| Ret | PROTO-ONCOGENE TYROSINE KINASE | AF042830 | 0.6147 | 0.0162 |
| | RECEPTOR RET | | | |
| Hpn | HEPSIN | NM_017112 | 0.7204 | 0.0253 |
| Utrn | UTROPHIN | NM_013070 | 3.1316 | 0.0348 |
| Avpr2 | ARGININE VASOPRESSIN RECEPTOR 2 | NM_019136 | 1.6044 | 0.0399 |
| Grm3 | GLUTAMATE RECEPTOR, METABOTROPIC 3 | M92076 | 0.6892 | 0.0427 |
| Olr1468 | OLFACTORY RECEPTOR 1468 | M64392 | 0.5905 | 0.0478 |

Orphan genes

| | | | | |
|---|---|---|---|---|
| Sp1 | SP1 TRANSCRIPTION FACTOR | CB730302 | 0.4620 | 0.0014 |
| Has3 | HYALURONAN SYNTHASE 3 | NM_172319 | 0.6042 | 0.0043 |
| Rassf1 | RAS ASSOCIATION (RALGDS/AF-6) DOMAIN | AI103943 | 2.2816 | 0.0190 |
| | FAMILY 1 | | | |
| Wnt2b | WINGLESS-TYPE MMTV INTEGRATION SITE | AF204873 | 0.6391 | 0.0232 |
| | FAMILY, MEMBER 2B | | | |
| Slit1 | SLIT HOMOLOG 1 (DROSOPHULA) | AB073215 | 0.6607 | 0.0235 |
| Ing4 | INHIBITOR of GROWTH FAMILY, MEMBER 4 | AA850369 | 2.1524 | 0.0478 |
| Muc13 | MUCIN 13, EPITHELIAL TRANSMEMBRANE | CB577563 | 0.5201 | 0.0488 |

In general, these data suggest that a fish oil/pectin diet altered gene expression profiles for genes that are associated with the cell membrane. Fish oil/pectin diet altered expression of genes involved in cell adhesion and receptor activity, which are almost exclusively located in cell membranes. Cell adhesion and receptor activity are positive regulators of the Wnt/β-catenin signaling pathway, suggesting that the suppression of gene expression involving in the pathway by fish oil/pectin diet intake might mitigate the enhanced risks of carcinogenesis by radiation treatment. The data show that not only was gene expression involved in apoptosis altered by fish oil/pectin diet, but also genes related to proliferation were down-regulated.

a. Cell Adhesion Genes

Thirteen (13) cell adhesion genes were differentially expressed following a fish oil/pectin diet. Of these thirteen, three (3) genes were up-regulated (Aoc3, Cdc42 and Lpp), and ten (10) genes were down-regulated (Cd36, Cd47, Cd97, Cdh2, Col5a3, Kifap3, nell2, Robo1, Src and Tspan1). Cell adhesion molecules are believed involved in the development and progression of colon cancer, especially in the step of cancer cell metastasis. In contrast to the above discussed data regarding radiation-induced differential gene expression, few genes responding to dietary changes are associated with the Wnt/β-catenin signaling pathway.

The cell adhesion molecule having the most significant response to a fish oil/pectin diet was Kinesin-associated protein 3 (Kifap3). Kifap3 is believed to be a small G protein GDP dissociation stimulator, which transports the tumor suppressor adenomatous polyposis colon protein (APC) and cleaves a Cdh2/β-catenin complex resulting in the translocation of β-catenin to the nucleus. Jimbo et al., “Identification of a link between the tumour suppressor APC and the kinesin superfamily” Nat Cell Biol 4:323-327 (2002).

The Src gene is believed to encode a proto-oncogene tyrosine-protein kinase, and its activation is evident in 80% of human colon cancer. It has been reported that phosphorylation of β-catenin by Src dissociates the cell-cell adhesion complex, such as E-cadherin or N-cadherin (Cdh2), inducing the translocation of β-catenin to the nucleus. Kopetz S., “Targeting SRC and epidermal growth factor receptor in colorectal cancer: rationale and progress into the clinic” Gastrointest Cancer Res 1:S37-S41 (2007); and Qi et al., “Involvement of Src family kinases in N-cadherin phosphorylation and beta-catenin dissociation during transendothelial migration of melanoma cells” Mol Biol Cell 17:1261-1272 (2006). The binding of Slit to its receptor, Roundabout, axon guidance receptor homolog 1 (Robo1) forms a complex with N-cadherin-associated β-catenin, and induce β-catenin dissociation and nuclear translocation via the inactivation of N-cadherin. Rhee et al., “Cables links Robo-bound Abl kinase to N-cadherin-bound beta-catenin to mediate Slit-induced modulation of adhesion and transcription” Nat Cell Biol 9:883-892 (2007).

The data presented herein show that Wnt/β-catenin signaling pathway regulators were down-regulated in the fish oil/pectin group. For example, the expression of Wnt2b, believed to play a role in the Wnt/β-catenin signaling pathway, was reduced in the fish oil/pectin group. Further, the data suggest that the increased cdc42 gene expression subsequent to fish oil/pectin diet intake might induce the binding between β-catenin and E-cadherin thereby preventing β-catenin release. FIG. 15; and Fukata et al., “cdc42 and Rac1 regulate the interaction of IQGAP1 with beta-catenin” J Biol Chem 274:26044-26050 (1999).

Several genes were down-regulated by a fish oil/pectin diet that have been previously reported to be positively correlated with colon cancer by increasing cell proliferation, increasing angiogenesis and/or mediating colorectal cancer metastasis included, but were not limited to, CD36, CD47, CD97, Neural epidermal growth factor-like 2 (Nell2) and Tetraspanin 1 (Tspan1). Gutierrez L. S., “The Role of Thrombospondin 1 on Intestinal Inflammation and Carcinogenesis” Biomark Insights 2008:171-178 (2008); Steinert et al., “Expression and regulation of CD97 in colorectal carcinoma cell lines and tumor tissues” Am J Pathol 161:1657-1667 (2002); Kuroda et al., “Biochemical characterization and expression analysis of neural thrombospondin-1-like proteins NELL1 and NELL2” Biochem Biophys Res Commun 265:79-86 (1999); and Chen et al., “TSPAN1 protein expression: a significant prognostic indicator for patients with colorectal adenocarcinoma” World J Gastroenterol 15:2270-2276 (2009).

“Receptor activity” as detected by GO analysis was over-represented in groups fed a fish oil/pectin diet (p=0.0352). For example, a fish oil/pectin diet caused up-regulation of Avpr2 and Utrn, and down-regulation of Cd36, Cd79b, Cd97, Gabrr2, Gfra3, Glp1r, Grik5, Grm3, Grm7, Hpn, Htr2c, Kiss1r, Il12rb2, Ogfrl1, Olr1468, Olr1654, Ret, Robo1, Pthr2, Rxra, Sectm1a and Thrap1.

Glial cell line-derived neurotrophic factor (GDNF) family receptor alpha (Gfra) is believed to form a signaling receptor complex with RET tyrosine kinase receptor. Although it is not necessary to understand the mechanism of an invention, it is believed that RET activates downstream growth pathways such as MAPK pathway, and when such signaling activation is uncontrolled, RET signaling may be associated with the development of human colon, pancreatic and breast cancer, especially papillary thyroid cancer, and Hirschsprung's disease, congenital agangliosis of the colon. Castellone et al., “Dysregulated RET signaling in thyroid cancer” Endocrinol Metab Clin North Am 37:363-374 (2008).

Emerging evidence suggests that glutamate and its receptors have possible roles in cancer, and glutamate antagonists limit tumor growth. Stepulak et al., “Expression of glutamate receptor subunits in human cancers” Histochem Cell Biol 132:435-445 (2009). The data presented herein, show that the expression of glutamate receptors, Grik5, Grm3 and Grm7, were down-regulated by the fish oil/pectin diet.

Glucagon-like peptide 1 receptor (Glp1r) was recently shown to be overexpressed in endocrine tumors of the gut, and it may play a role in the endocrine activity, growth, and differentiation of tumor cells. Korner et al., “GLP-1 receptor expression in human tumors and human normal tissues: potential for in vivo targeting” J Nucl Med 48:736-743 (2007).

5-hydroxytryptamine (serotonin) receptor 2C (Htr2c) is a G protein receptor for serotonin, which acts as an enhancer of angiogenesis in colon cancer. Nocito et al., “Serotonin regulates macrophage-mediated angiogenesis in a mouse model of colon cancer allografts” Cancer Res 68:5152-5158 (2008).

Interleukin 12 receptor, beta 2 (Il12rb2) promotes cell proliferation via the activation of the JAK2/STAT4 pathway, and inhibits FAS-dependent apoptosis via inhibition of caspase 3 activity in a number of infectious diseases such as Crohn's disease. Also, in relation to antitumor activity, it activates innate and adaptive effector immune mechanisms, and inhibits angiogenesis Airoldi et al., “Endogenous IL-12 triggers an antiangiogenic program in melanoma cells” Proc Natl Acad Sci USA 104:3996-4001 (2007). The data show that a fish oil/pectin diet resulted in the down-regulation of these oncogenes.

“Ion transmembrane transporter activity” as detected by GO analysis was found to be overexpressed following administration of a fish oil/pectin diet. Ion transmembrane transporters such as voltage-gated potassium channels (VGPCs) have been shown to be directly implicated in cell proliferation by controlling cell cycle progression. Further, it has been reported that several ion transmembrane channels are overexpressed in tumors and are therefore candidate targets for anticancer therapies. Felipe et al., “Potassium channels: new targets in cancer therapy” Cancer Detect Prey 30:375-385 (2006). The data presented herein is consistent with these reports wherein the expression of Kcnab1 and Kcnq2 were down-regulated by a fish oil/pectin diet. It has been reported that cell cycle progression may also be regulated by CDKs and cyclins that are transcriptionally and post-transcriptionally controlled to provide regulatory flexibility at the level of input. Although it is not necessary to understand the mechanism of an invention, it is believed that fish oil with butyrate beneficially modulates p21Waf1/Cip1 gene expression, a CDK inhibitor.

A fish oil/pectin diet decreased expression of Sp1 oncogene and colon cancer markers such as Hyaluronan synthase 3 and/or Mucin 13. Dong et al., “Sp1 upregulates expression of TRF2 and TRF2 inhibition reduces tumorigenesis in human colorectal carcinoma cells” Cancer Biol Ther 8:2166-2174 (2009); Bullard et al., “Hyaluronan synthase-3 is upregulated in metastatic colon carcinoma cells and manipulation of expression alters matrix retention and cellular growth” Int J Cancer 107:739-746 (2003); and Walsh et al., “The MUC13 cell surface mucin is highly expressed by human colorectal carcinomas” Hum Pathol 38:883-892 (2007).

A fish oil/pectin diet increased expression of tumor suppressor genes such as Ras association (Ralgds/AF-6) domain family 1 (Rassf1) and Inhibitor of growth family, member 4 (Ing4). Lauer et al., “Impairment of peroxisomal biogenesis in human colon carcinoma” Carcinogenesis 20:985-989 (1999); and Unoki et al., “Reviewing the current classification of inhibitor of growth family proteins” Cancer Sci 100:1173-1179 (2009).

In general, the data presented herein demonstrate that expression of several receptor genes were down-regulated in fish oil/pectin diet group. The data suggest that β-catenin not only increases transcription of Wnt target genes but also cadherin-mediated cell adhesion. Although it is not necessary to understand the mechanism of an invention, it is believed that phosphorylation of β-catenin by Src tyrosine kinases dissociates the cell-cell adhesion complex, resulting in an increase of cytoplasmic β-catenin. Nelson et al., “Convergence of Wnt, beta-catenin, and cadherin pathways” Science 303:1483-1487 (2004). Such a dissociation is further believed to result in β-catenin entering the nucleus thereby activating target gene transcription. Although it is not necessary to understand the mechanism of an invention, it is believed that a downregulation of cell adhesion related genes by the fish oil/pectin diet might be related to suppression of Wnt/β-catenin signaling activity.

3. Interactions of Radiation and Diet

A functional analysis of the sixty-four (64) differentially expressed genes that were co-correlated with radiation and a fish oil/pectin diet identified at least two (2) GO biological processes including, but not limited to, the phospholipid biosynthetic process (p=0.0420). See, Table 10.

TABLE 10
Functional categories with significant enrichment of genes differentially expressed by
the radiation and the diet interaction.

| | | | | | | | |
|---|---|---|---|---|---|---|---|
| | | | | FPR | | | p- |
| Symbol | Gene name | GenBank ID | CCR (−) | (−) | CCR (+) | FPR (+) | value |

GO Biological Processes
Phospholipid biosynthetic process (p = 0.0420)

| | | | | | | | |
|---|---|---|---|---|---|---|---|
| Pcyt1b | PHOSPHATE CYTIDYLYL- | NM_173151 | 0.7047 | 0.6342 | 0.6625 | 1.1503 | 0.0199 |
| | TRANSFERASE 1, | | | | | | |
| | CHOLINE, BETA ISOFORM | | | | | | |
| Pcyt2 | PHOSPHATE CYTIDYLYL- | NM_053568 | 19.7393 | 8.4737 | 8.7241 | 16.3362 | 0.0257 |
| | TRANSFERASE 2, | | | | | | |
| | ETHANOLAMINE | | | | | | |

Cell-cell signaling (p = 0.0447)

| | | | | | | | |
|---|---|---|---|---|---|---|---|
| Ntrk2 | NEUROTROPHIC TYROSINE | BF391024 | 0.7195 | 2.0378 | 1.3803 | 1.0389 | 0.0157 |
| | KINASE, | | | | | | |
| | RECEPTOR, TYPE 2 | | | | | | |
| Ap3m2 | ADAPTOR-RELATED PROTEIN | NM_133305 | 0.9883 | 7.3360 | 0.9979 | 1.1712 | 0.0224 |
| | COMPLEX 3, MU 2 SUBUNIT | | | | | | |
| Dlgap4 | DISCS, LARGE HOMOLOG- | NM_173145 | 4.0925 | 5.0912 | 11.1193 | 2.2176 | 0.0225 |
| | ASSOCIATED PROTEIN 4 | | | | | | |
| | (DROSOPHILA) | | | | | | |
| Slc6a7 | SOLUTE CARRIER FAMILY 6 | NM_053996 | 1.0274 | 1.4814 | 1.6256 | 1.1423 | 0.0413 |
| | (NEUROTRANSMITTER | | | | | | |
| | TRANSPORTER, L-PROLINE), | | | | | | |
| | MEMBER 7 | | | | | | |
| Ide | INSULIN DEGRADING ENZYME | BF524009 | 1.8856 | 0.8932 | 1.2527 | 1.4671 | 0.0454 |
| Ghrl | GHRELIN PRECURSOR | NM_021669 | 1.1004 | 1.4641 | 1.6737 | 0.9559 | 0.0460 |
| Csp | CYSTEINE STRING PROTEIN | BE109704 | 7.6688 | 8.7120 | 20.4780 | 4.9417 | 0.0492 |

Orphan genes

| | | | | | | | |
|---|---|---|---|---|---|---|---|
| Mvp | MAJOR VAULT PROTEIN | NM_022715 | 16.4270 | 8.1455 | 4.7966 | 12.1594 | 0.0101 |
| Rsg12 | REGULATOR OF G-PROTEIN | NM_019339 | 1.3880 | 1.5347 | 2.1067 | 1.2631 | 0.0237 |
| | SIGNALING 12 | | | | | | |
| Atg7 | AUTOPHAGY 7-LIKE | AW920947 | 0.7047 | 1.1835 | 1.2649 | 0.7830 | 0.0275 |
| | (S. CEREVISIAE) | | | | | | |
| Ccr6 | CHEMOKINE (C-C MOTIF) | AI598324 | 0.9599 | 2.2894 | 9.6934 | 1.8764 | 0.0425 |
| | RECEPTOR 6 | | | | | | |

Phosphatidylcholine (PC) and phosphatidylethanolamine (PE) have been suggested to be major phospholipids in membranes, wherein the PC/PE ratio is used by some as a modulator of membrane integrity. For example, a decreased PC/PE ratio correlates with a loss of membrane integrity leading to the release of cellular contents and increasing the influx of extracellular components. Li et al., “The ratio of phosphatidylcholine to phosphatidylethanolamine influences membrane integrity and steatohepatitis” Cell Metab 3:321-331 (2006). It has been reported that lipid profiles in rats fed a diet high in n-6 polyunsaturated fatty acids reflect a decreased PC/PE ratio, because PE increases are relatively higher than increases of PC.

Pcyt2 expression was decreased in rats fed a fish oil/pectin diet but was unaffected following radiation exposure (supra). However, Pcyt1b expression was affected by the combination of radiation exposure and fish oil/pectin diet. The data suggest that PC/PE ratios calculated using RNA expression levels were increased by radiation exposure, fish oil/pectin diet, and their combination. In the irradiated group, the fish oil/pectin diet couldn't affect on the PC/PE ratio, and these treatments might modulate the membrane damages caused by the combination of radiation and the chemical carcinogen.

Radiation exposure induced the overexpression of neurotrophic tyrosine kinase, receptor type2 (Ntrk2), Regulator of g-protein signaling 12 (Rgs12) and chemokine (C-C motif) receptor 6. These genes possibly play a role in activating ERK signaling, inhibiting apoptosis, promoting proliferation and invasion Yu et al., “Overexpression of TrkB promotes the progression of colon cancer” APMIS 118:188-195 (2010); Brand et al., “Cell differentiation dependent expressed CCR6 mediates ERK-1/2, SAPK/JNK, and Akt signaling resulting in proliferation and migration of colorectal cancer cells” J Cell Biochem 97:709-723 (2006); and Willard et al., “Selective role for RGS12 as a Ras/Raf/MEK scaffold in nerve growth factor-mediated differentiation” EMBO J 26:2029-2040 (2007). However, the data presented herein show that a fish oil/pectin diet down-regulated the gene expressions of these same genes. As a result, the data suggests that the interaction between radiation and diet modulates membrane integrity.

In one embodiment, the present invention contemplates a method comprising a synergistic suppression of radiation enhanced colon carcinogenesis following down-regulation of ERK signaling activators due to the combined effects of radiation and a fish oil/pectin diet. Although it is not necessary to understand the mechanism of an invention, it is believed that ERK signaling activators such as Ntrk2, Rgs12 and Ccr6 were up-regulated in expression following a corn oil/cellulose diet in irradiated rats, but were down-regulated in a fish oil/pectin irradiated group. Furthermore, the gene expression patterns involved in modulating membrane integrity and ERK signaling pathway were altered by radiation and diet interaction. Gene expressions of Ntrk2, Rgs12 and Ccr6 activating the ERK signaling pathway, which enhances cancer cell proliferation, were induced in corn oil/cellulose irradiated groups, but decreased in fish oil/pectin irradiate groups. The present data suggest that fish oil/pectin diet has more effects of protection against radiation enhanced colon cancer as compared to carcinogen-only (i.e., for example, AOM) induced colon cancer.

### V. Pharmaceutical Formulations

The present invention further provides pharmaceutical compositions (e.g., comprising the dietary components described above). In some embodiment, various dietary oils and/or polymers may be incorporated into various binders. Such binders may include, but are not limited to, meat, vegetables, fruits, dairy products, cereals, starches, proteins etc. Dietary oils may include, but are not limited to, fish oils, olive oils, nut oils, seed oils etc. Polymers useful within pharmaceutical formulations of the present invention, include, but are not limited to, pectins, sugars, carbohydrates, etc.

The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention, the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### VI. Detection Methodologies

A. Detection of Nucleic Acids

mRNA expression may be measured by any suitable method, including but not limited to, those disclosed below.

In some embodiments, RNA is detected by Northern blot analysis. Northern blot analysis involves the separation of RNA and hybridization of a complementary labeled probe.

In other embodiments, RNA expression is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific nucleic acid (e.g., RNA) sequences by using structure-specific enzymes to cleave a complex formed by the hybridization of overlapping oligonucleotide probes.

In still further embodiments, RNA (or corresponding cDNA) is detected by hybridization to a oligonucleotide probe. A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

B. Sequencing of Nucleic Acids

The method most commonly used as the basis for nucleic acid sequencing, or for identifying a target base, is the enzymatic chain-termination method of Sanger. Traditionally, such methods relied on gel electrophoresis to resolve, according to their size, wherein nucleic acid fragments are produced from a larger nucleic acid segment. However, in recent years various sequencing technologies have evolved which rely on a range of different detection strategies, such as mass spectrometry and array technologies.

One class of sequencing methods assuming importance in the art are those which rely upon the detection of PPi release as the detection strategy. It has been found that such methods lend themselves admirably to large scale genomic projects or clinical sequencing or screening, where relatively cost-effective units with high throughput are needed.

Methods of sequencing based on the concept of detecting inorganic pyrophosphate (PPi), which is released during a polymerase reaction have been described in the literature for example (WO 93/23564, WO 89/09283, W098/13523 and WO 98/28440). As each nucleotide is added to a growing nucleic acid strand during a polymerase reaction, a pyrophosphate molecule is released. It has been found that pyrophosphate released under these conditions can readily be detected, for example enzymically e.g. by the generation of light in the luciferase-luciferin reaction. Such methods enable a base to be identified in a target position and DNA to be sequenced simply and rapidly whilst avoiding the need for electrophoresis and the use of labels.

At its most basic, a PPi-based sequencing reaction involves simply carrying out a primer-directed polymerase extension reaction, and detecting whether or not that nucleotide has been incorporated by detecting whether or not PPi has been released. Conveniently, this detection of PPi-release may be achieved enzymatically, and most conveniently by means of a luciferase-based light detection reaction termed ELIDA (see further below).

It has been found that dATP added as a nucleotide for incorporation, interferes with the luciferase reaction used for PPi detection. Accordingly, a major improvement to the basic PPi-based sequencing method has been to use, in place of dATP, a dATP analogue (specifically dATP.alpha.s) which is incapable of acting as a substrate for luciferase, but which is nonetheless capable of being incorporated into a nucleotide chain by a polymerase enzyme (WO98/13523).

Further improvements to the basic PPi-based sequencing technique include the use of a nucleotide degrading enzyme such as apyrase during the polymerase step, so that unincorporated nucleotides are degraded, as described in WO 98/28440, and the use of a single-stranded nucleic acid binding protein in the reaction mixture after annealing of the primers to the template, which has been found to have a beneficial effect in reducing the number of false signals, as described in WO00/43540.

C. Detection of Protein

In other embodiments, gene expression may be detected by measuring the expression of a protein or polypeptide. Protein expression may be detected by any suitable method. In some embodiments, proteins are detected by immunohistochemistry. In other embodiments, proteins are detected by their binding to an antibody raised against the protein. The generation of antibodies is described below.

Antibody binding may be detected by many different techniques including, but not limited to, (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference.

D. Remote Detection Systems

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, wherein the information is provided to medical personal and/or subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

E. Detection Kits

In other embodiments, the present invention provides kits for the detection and characterization of proteins and/or nucleic acids. In some embodiments, the kits contain antibodies specific for a protein expressed from a gene of interest, in addition to detection reagents and buffers. In other embodiments, the kits contain reagents specific for the detection of mRNA or cDNA (e.g., oligonucleotide probes or primers). In preferred embodiments, the kits contain all of the components necessary to perform a detection assay, including all controls, directions for performing assays, and any necessary software for analysis and presentation of results.

### Experimental

### Example I

### Animals, Diets, and Radiation Exposure

The animals used in this study were a subset of a larger study (560 rats) for the development of gene expression profiles. In this particular study, colonic mucosal and exfoliated cells were characterized for radiation enhancing effects at three time points of colon carcinogenesis and assessed the anti-carcinogenic effect of fish oil/pectin diet. The basic study comprises variations of a 2×2×3 factorial design with two types of diet (fish oil/pectin or corn oil/cellulose), two kinds of radiation treatments (irradiation or non-irradiation), and three feces collection time points (i.e., for example, 7, 14, and 28 weeks). See, FIG. 1, FIG. 6, and FIG. 7. The animal use protocol was approved by the Institutional Animal Care and Use Committee (IACUC) of Texas A&M University and conformed to National Institutes of Health (NIH) guidelines.

Male Sprague-Dawley rats (3 week-old) were supplied from Harlan Teklad (Madison, Wis.). The animals were kept in suspended cages in a room controlled at a temperature of 23±2° C., a humidity of 55±5% and a 12 h light/dark cycle. The rats had free access to food and water. After one week of adaptation, rats were assigned to treatment groups so that initial body weights were similar among treatment groups.

Experimental diets were fed to all rats for 3 weeks. Rats were provided one of the two experimental diets, which differed only in fat and dietary fiber. The experimental diet contained 11.5% fish oil (Vacuum deodorized menhaden fish oil; 18.2% EPA and 11.3% DHA; Degussa, Waukesha, Wis.) and 3.5% corn oil (55.4% Linoleic acid; Degussa, Waukesha, Wis.) as the source of fat, and 6% pectin (Citrus pectin; Danisco Cultor, Kans.) as the source of dietary fiber compared to the basal diet that contained 15% corn oil and 6% a-cellulose. The diets provided 30% of energy from lipid, which is consistent with current dietary guidelines for humans, and the amount of fiber in the diet corresponded to a 30 g per day dietary fiber intake in humans, which is within the recommended range. Antioxidants were supplemented to protect against fatty acid oxidation while maintaining equivalent antioxidant levels in both diets. Vanamala et al., “Dietary fish oil and pectin enhance colonocyte apoptosis in part through suppression of PPARdelta/PGE2 and elevation of PGE3” Carcinogenesis 29:790-796 (2008).

Then, half of the rats were exposed to heavy ion irradiation; 1 Gy, 1 GeV/nucleon Fe ions at Brookhaven National Laboratory (Upton, N.Y.). A 1 Gy dose was chosen since it approximates the exposure level that astronauts might be expected to experience to during an exemplary 3-year deep space exploration mission (i.e., for example, a round-trip Mars expedition). Cucinotta et al., “Cancer risk from exposure to galactic cosmic rays: implications for space exploration by human beings” Lancet Oncol 7:431-435 (2006). At 10 and 17 days post-irradiation (or sham irradiation) all rats were given subcutaneous injections of 15 mg/kg azoxymethane (AOM; Sigma Chemical Co., St Louis, Mo.).

### Example II

### RNA Extraction and Microarray Analysis

At time of sample collection described in Example I, fecal pellets were immediately put into the Ambion lysis solution (Austin, Tex.), homogenized with a pestle and stored at −80° C. Using extracted and isolated RNA from the collected fecal matter, microarray data were constructed, evaluated, and statistically analyzed using a mixed model ANOVA procedure.

Fecal poly (A)+ RNA was subsequently isolated from feces using the mTRAP Maxi® kit (Active Motif, Carlsbad, Calif.) according to the manufacturer's protocol. Isolated poly (A)+ RNA quality and quantity were determined using an Agilent Bioanalyzer 2100 using the pico assay.

A Rat Whole Genome Bioarray (GE CodeLink® system, Piscataway, N.J.) containing 35,129 gene probes was used to examine differential gene expression between the diet and radiation treatment groups. All reagents were provided in the GE CodeLink® expression assay kits. Target preparation and hybridization processing were in accordance with methods as previously reported. Davidson et al., “Chemopreventive n-3 polyunsaturated fatty acids reprogram genetic signatures during colon cancer initiation and progression in the rat” Cancer Res 64:6797-6804 (2004). Images were captured on an Axon GenePix® Scanner (Arlington, Tex.) and analyzed using CodeLink® Expression Analysis Software.

### Example III

### Differential Gene Expression During Colorectal Cancer Development

In accordance with Example I and Example II, rats were injected with a colorectal cancer-specific carcinogen (azoxymethane, AOM, 2×, 15 mg/kg BW), to induce colon cancer development. Fecal matter was collected during Week 7, Week 14, and Week 28 following AOM injection.

Using extracted and isolated RNA from the collected fecal matter, microarray data were constructed, evaluated, and statistically analyzed using a mixed model ANOVA procedure as discussed above. See, FIG. 8. Genes exhibiting differential expression underwent Gene Ontology (GO) enrichment analyses (i.e., using the DAVID software program). See. FIG. 2.

### Example IV

### Data Normalization and Statistical Analysis

The data were normalized to reduce loss of information or potentially biased outcomes. Because certain probe information was compromised due to partial mRNA degradation, a two-stage semiparametric normalization method was used. For example, a location-scale transformation and a robust inclusion step were performed to roughly match arrays to the normal distribution. A non-parametric estimated non-linear transformation was applied to remove the potential intensity-based biases within the same treatment. Liu et al., “A two-stage normalization method for partially degraded mRNA microarray data” Bioinformatics 21:4000-4006 (2005).

Following normalization, the data were analyzed using a linear mixed model ANOVA procedure with a standard false discovery rate (FDR) method. Benjamini et al., “Controlling the false discovery rate: a practical and powerful approach to multiple testing” J. R. Statist. Soc. 57:289-300 (1995). This multiple testing correction adjusts individual p-values to consider the joint distribution of the test statistics for each of the hypothesis tests, and outcomes with an adjusted p-value less than 0.05 were selected for further observation. The effect of irradiation, diet, and their interaction were examined by this test.

### Example V

### Enrichment Analysis

Enrichment analysis data mining tools, such as Gene Ontology (GO), comprise strategies to systematically cluster an abundance of genes to the associated biological annotation under the study of radiation exposure and dietary intervention. Huang et al., “Systematic and integrative analysis of large gene lists using DAVID bioinformatics resources” Nat Protoc 4:44-57 (2009). To identify potential pathways that may be altered by irradiation, diet and/or their interaction, normalized gene data collected herein was further subjected to GO analysis.

GO analysis was facilitated by an internet-based software research tool known as DAVID (e.g., Database for Annotation, Visualization and Integrated Discovery) which comprises a bioinformatics resource and GO mapping tools. david.abcc.ncifcrf.gov; and ebi.ac.uk/ego/GmultiTerm, respectively. In general, DAVID comprises analytic tools aimed at systematically extracting biological meaning from large sets of genes. The results from DAVID were applied on a GO mapping program to visualize the hierarchical structure of GO terms. The specific genes involved in the endpoint terms in the GO map were selected, because the alteration of the endpoint terms might cause upper pathways to change. Additional information of genes was derived from literature searches and DAVID to gain insights of their contribution to the colon carcinogenesis.

### Example VI

### Quantitative Real-Time Polymerase Chain Reaction

To validate alterations in gene expression identified using microarrays, real-time PCR was performed on poly (A)+ RNA from same rats. Using selected genes, primers were designed using commercially available software (Primer Express, Applied Biosystems, Foster City, Calif.).

For example, three of the selected genes used the following primer pairs:

| | | |
|---|---|---|
| Dusp5 | | |
| Forward: | AGAAAGTTGTCAGACCCTAGACCAAA | (SEQ ID NO: 1) |
| Reverse: | GGTAGGGAGGGAAACATTGTCACA | (SEQ ID NO: 2) |
| Src | | |
| Forward: | AGGACAGGTTGAGGCTGGTACA | (SEQ ID NO: 3) |
| Reverse: | GGTAGAGTGGGTTGAGGTTGGAAA | (SEQ ID NO: 4) |
| Pyct2 | | |
| Forward: | CAGCAGGCTCTCAGTCCTTTCC | (SEQ ID NO: 5) |
| Reverse: | TCCAGTCACAGGTGCCGTCT | (SEQ ID NO: 6) |

Briefly, partially degraded fecal poly (A)+ RNA was amplified and converted to cDNA using WT-Ovation™ FFPE RNA Amplification System (NuGEN, San Carlos, Calif.). The quantitative real-time PCR assay was performed on an ABI 7700 system (Applied Biosystems, Foster City, Calif.) using SYBR green kits and the PCR primers for Dusp, Src and Pcyt2 genes.

### Example VII

### Statistics

The two diet groups (Corn oil/cellulose and fish oil/pectin) and two radiation treatments (non-irradiated and irradiated rats) represent a 2×2 factorial design. Tumor incidence data were analyzed using the FREQ procedures in SAS. Number of high multiplicity aberrant crypt foci (HMACF) and qRT-PCR results were analyzed using the General Linear Models procedure of SAS. Differences were considered significant when p<0.05.

### Example VIII

### Animals, Diets and Dietary Chemoprotection

Male Sprague-Dawley rats (Harlan Teklad) were used to study the chemoprotective effect of FO/P at the initiation, ACF, and tumor stages of colon cancer. The animal use protocol was approved by the University Animal Care Committee of Texas A&M University and conformed to National Institutes of Health (NIH) guidelines.

### Animals

Rats were housed individually in a temperature and humidity controlled animal facility with a 12 h light/dark cycle. After 1 week of acclimation and 31 days receiving the experimental diets, rats were injected with azoxymethane (AOM; Sigma, St Louis, Mo., 15 mg/kg body weight). For the initiation stage analyses, 22 rats were terminated 24 hours after AOM injection. At termination, fresh fecal material was collected and placed into RNA isolation solution for microarray analysis and colon samples were collected and processed as described below. Rats used for the ACF stage (n=40) were raised using the same diet and treatment conditions with the exception that they received a second AOM injection 1 week after the first injection. Seven weeks after the second AOM injection, rats were terminated and colon samples were collected. Rats for the tumor stage analyses (n=80) were raised using the same diet and treatment conditions as the ACF stage rats, except they were terminated at 31 week after the second AOM injection. Feces from the tumor stage rats were collected at 7 and 28 weeks after the second AOM injection, and colon samples were collected at termination.

### Diet

Rats were assigned to receive a diet containing either FO/P or CO/C. Davidson et al., “Chemopreventive n-3 polyunsaturated fatty acids reprogram genetic signatures during colon cancer initiation and progression in the rat” Cancer Research 64:6797-804 (2004). All diets contained oils at 15% by weight and 30% of calories. The two lipid sources differed in fatty acid compositions; FO contains higher amounts of eicosapentaenoic acid (EPA, 20:5, n-3) and docosahexanoic acid (DHA, 22:6, n-3) than CO, which has higher amounts of linoleic acid (LA, 18:2, n-6). The fish oil diet included 3.5 g corn oil/100 g diet to prevent essential fatty acid deficiency. The amount of fiber in the diet was 6% by weight, which is equivalent to 30 g/d for humans. Fiber sources had differences in the fermentability; pectin is highly fermentable whereas cellulose is poorly fermented. Citrus pectin was obtained from Danisco Cultor (New Century, Kans.) and bulk vacuum-deodorized menhaden fish oil was obtained from Degussa lipids (Degussa, Waukesha, Wis.). The antioxidant levels in the diets were balanced by including 15 mg α-tocopherol, 14 mg γ-tocopherol, and 5 mg tertiary butylhydroquinone/100 g diet in the FO/P diet and 19 mg tertiary butylhydroquinone/100 g diet in the CO/C diet. Animals were provided with fresh diet daily to prevent lipid oxidation. Food and water were provided ad libitum.

### Tissue Collection

Rats were sacrificed by CO₂ overdose and cervical dislocation. The colon was resected and 1 cm of the distal colon was fixed in 4% paraformaldehyde (PFA) and another 1 cm of distal colon was used for 70% ethanol fixation. At initiation, the remaining colon was scraped and processed for mRNA analysis and stored at −80° C. At the ACF stage, the remaining colon was divided longitudinally and half was used for ACF scoring and mucosa from the other half was used for mRNA analysis. Tissues from the tumor stage were evaluated for tumor incidence. Tumors were removed and fixed as described below, while the remaining tissues were scraped to remove the mucosa, which was processed for mRNA analysis. All samples for mRNA analyses were homogenized on ice in denaturation solution (Ambion, Austin, Tex.) and frozen at −80° C. until the RNA was isolated.

RNA Isolation from Fecal and Mucosal Samples

To enrich the level of eukaryotic mRNA in the fecal samples, poly (A)+ RNA was isolated from total RNA using oligo dT cellulose micro spin columns and the mTRAP Maxi kit (Active Motif, Carlsbad, Calif.). Davidson et al., “Noninvasive detection of putative biomarkers for colon cancer using fecal messenger RNA” Cancer Epidemiol Biomarkers Prey. 4:643-647 (1995). Mucosal RNA was extracted from scraped mucosa as described previously using the Totally RNA kit (Ambion, Austin, Tex.) followed by DNase treatment. Davidson et al., “Chemopreventive n-3 polyunsaturated fatty acids reprogram genetic signatures during colon cancer initiation and progression in the rat” Cancer Research. 64:6797-6804 (2004). Both mucosal RNA and fecal poly (A)+ RNA were analyzed on an Agilent Bioanalyzer 2100 (Agilent, Palo Alto, Calif.) using the pico assay to assess mRNA quality and quantity.

### Microarray Data Acquisition

Fecal poly (A)+ RNA and mucosal RNA were used to monitor gene expression using CodeLink Rat Whole Genome Bioarrays (GE, Piscataway, N.J.) containing 35,129 genes probes. cRNA synthesis was performed as per manufacturer's instructions (the CodeLink expression assay kit) using 10 μg of mucosal RNA and 1 μg of fecal poly (A)+ RNA. Purified and fragmented cRNA was hybridized to a Rat Whole Genome Bioarray in an Innova 4080 shaking incubator (New Brunswick, Edison, N.J.) at 300 rpm. After hybridization, the arrays were processed as described above. Images of processed arrays were captured on an Axon GenePix Scanner (Arlington, Tex.).

### High Multiplicity Aberrant Crypt Foci (HM ACF) Assay

Colon samples were collected from rats 7 weeks after the 2nd AOM injection. Briefly, colons were opened and placed flat within folded Whatman #1 paper, followed by fixation in 70% ethanol for 24 h. To identify aberrant crypts, the tissue was stained in a 0.5% solution of methylene blue for 45 s. The total number of HM ACF (include four or more aberrant crypts per focus) was counted using a 40× objective. Vanamala et al., “Suppression of colon carcinogenesis by bioactive compounds in grapefruit” Carcinogenesis 27:1257-1265 (2006).

### Colon Cancer Incidence

Colons from rats terminated at 31 wk after the 2nd AOM injection were used to determine tumor incidence. Tumors were counted and tumor bearing tissues were fixed in 4% PFA for 4 h and embedded in paraffin blocks for histological examination. Tumor sections (4 μm) were stained with hematoxylin and eosin, and tumors were classified as adenomas or adenocarcinomas. Chang et al., “Predictive value of proliferation, differentiation and apoptosis as intermediate markers for colon tumorigenesis” Carcinogenesis 18:721-730 (1997).

### In Situ Apoptosis

Apoptosis was measured by the binding of TdT-mediated UTP-biotin nick end labeling (TUNEL assay) to fragmented pieces of DNA using sections (4 μm) of PFA-fixed, paraffin-embedded tissue. Apoptotic cells with condensed chromatin, apoptotic bodies and intense brown staining were counted in 50 crypt columns for each animal. The apoptotic index is 100 times the mean number of apoptotic cells per crypt column divided by the total number of cells per crypt column. Chang et al., “Predictive value of proliferation, differentiation and apoptosis as intermediate markers for colon tumorigenesis” Carcinogenesis 18:721-730 (1997).

### Colonocyte Proliferation

Cell proliferation was measured using the proliferating cell nuclear antigen (PCNA) assay. Sections (4 μm) of 70% ethanol-fixed, paraffin-embedded tissue were incubated with 150 PCNA monoclonal antibody (Signet Laboratories, Inc., Dedham, Mass.). Sections were incubated with biotinylated antimouse IgG (Vector Lab, Burlingame, Calif.) and then stained with diaminobenzidine tetrahydrochloride (DAB; Sigma) and counterstained with hematoxylin. PCNA containing nuclei show up as brown spots within crypt columns, indicating a proliferating cell. Twenty-five crypt columns were counted per animal and the number and proportion of cells per crypt column and proliferating cells per crypt column were determined.

### Statistical Analysis

Gene expression data for the fecal samples were normalized using a two-stage semi-parametric normalization method, which is specifically designed for data from partially degraded mRNA. Liu et al., “A two-stage normalization method for partially degraded mRNA microarray data” Bioinformatics (Oxford, England) 21:4000-4006 (2005). The data were analyzed in SAS using ANOVA initially to evaluate the diet effect (FO/P vs. CO/C) at each time point. To correct for multiple testing, a false discovery rate was applied with a linear mixed model ANOVA procedure. Benjamini Y. H., “Controlling the false discovery rate: a practical and powerful approach to multiple testing” J R Statist Soc B 57:289-300 (1995). Genes that were differentially expressed (P<0.05) between diets from each time point were used for functional categorization based on gene ontology (Database for Annotation, Visualization, and Integrated Discovery (DAVID) bioinformatics resources, david.abcc.ncifcrf.gov/). Huang et al., “Systematic and integrative analysis of large gene lists using DAVID bioinformatics resources” Nature Protocols 4:44-57 (2009). By importing the list of differentially expressed genes, this program identified Gene Ontology (GO) categories showing enrichment for genes in the list and the probability of that GO category being significantly affected by diet and time. GO categories were chosen for study within “biological process” with a filter of enrichment p-value less than 0.05.

Data were analyzed using ANOVA to determine the effect of diet (FO/P vs. CO/C) on apoptosis, high multiplicity ACF, and proliferative zone (SAS Institute Inc.). Colon tumor incidence was analyzed by Chi square analysis and reported as percentage of rats bearing tumors (SAS Institute Inc.).

We claim:

1. A method, comprising:
a) providing;
i) exfoliated colonocytes derived from a subject; and
ii) a gene expression array; and
b) extracting nucleic acid from said colonocytes;
c) contacting said extracted nucleic acid with said gene expression array under conditions that create a gene expression profile.

2. The method of claim 1, wherein said nucleic acid comprise ribonucleic acid.

3. The method of claim 2, wherein said ribonucleic acid comprises messenger ribonucleic acid.

4. The method of claim 1, wherein said nucleic acid comprise deoxyribonucleic acid.

5. The method of claim 1, wherein said exfoliated colonocytes are collected from fecal material.

6. The method of claim 1, wherein said subject has been exposed to radiation.

7. The method of claim 1, wherein said subject has consumed a fish oil/pectin diet.

8. The method of claim 1, wherein said gene expression profile comprises down-regulated activators of the Ras-PI3K/Akt signaling pathway.

9. The method of claim 1, wherein said gene expression profile comprises up-regulated Wnt/β-catenin pathway genes.

10. The method of claim 1, wherein said gene expression profile comprises down-regulated Wnt/β-catenin pathway genes.

11. The method of claim 1, wherein said gene expression profile comprises up-regulated post-translational modification genes.

12. The method of claim 11, wherein said post-translational modification comprises a chemical modification.

13. The method of claim 1, wherein said gene expression profile comprises down-regulated EPH receptor genes.

14. The method of claim 1, wherein said gene expression profile comprises down-regulated tyrosine kinase receptor genes.

15. The method of claim 1, wherein said gene expression profile comprises up-regulated nuclear translocation genes.

16. The method of claim 1, wherein said gene expression profile comprises down-regulated tyrosine kinase pathway genes.

17. The method of claim 1, wherein said gene expression profile comprises down-regulated cell adhesion genes.

18. The method of claim 1, wherein said gene expression profile comprises down-regulated cell cycle regulator genes.

19. The method of claim 1, wherein said gene expression profile comprises down-regulated cell proliferation genes.

20. The method of claim 1, wherein said gene expression profile comprises down-regulated signal transduction genes.

21. The method of claim 1, wherein said gene expression profile comprises up-regulated tumor suppressor genes.

22. The method of claim 1, wherein said gene expression profile comprises down-regulated tumor invasion genes.

23. The method of claim 1, wherein said gene expression profile comprises up-regulated mitotic arrest genes.

24. A method, comprising:
a) providing;
i) a subject at risk for the development of a colorectal disease; and
ii) a composition comprising a mixture of fish oil and pectin;
b) administering the composition to the subject under conditions such that the colorectal disease development is reduced.

25. The method of claim 24, wherein said disease development is prevented.

26. The method of claim 24, wherein said subject at risk comprises a genetic predisposition for the colorectal disease.

27. The method of claim 24, wherein said colorectal disease or disorder is selected from the group including, but not limited to, colorectal cancer, colon cancer, large bowel cancer, colonic polyps, anal cancer, general anal and rectal diseases, colitis, Crohn's disease, hemorrhoids, ischemic colitis, ulcerative colitis, diverticulosis, diverticulitis and irritable bowel syndrome.

28. The method of claim 24, wherein said subject is a human.

29. The method of claim 24, wherein said administering comprises dietary consumption.

30. The method of claim 24, wherein said disease development reduction results from increased colorectal cell apoptosis.

31. The method of claim 24, wherein said disease development reduction results from decreased colorectal cell proliferation.

32. The method of claim 30, wherein said increased apoptosis comprises an upregulation of genes associated with the Wnt/β-catenin pathways.

33. The method of claim 31, wherein said decreased cell proliferation comprises an upregulation of genes associated with the Wnt/β-catenin pathways.

34. The method of claim 30, wherein said increased apoptosis comprises a downregulation of tyrosine kinase related genes.

35. The method of claim 31, wherein said decreased cell proliferation comprises a downregulation of tyrosine kinase related genes.

36. The method of claim 35, wherein said tyrosine kinase related genes regulate Ras-PI3K/Akt pathways.

37. The method of claim 24, wherein said subject at risk has been exposed to a carcinogen.

38. The method of claim 24, wherein said subject at risk has been exposed to a radiation source.

39. The method of claim 38, wherein said radiation source comprises cosmic radiation.

40. The method of claim 38, wherein said radiation source comprises a medical device.

41. The method of claim 38, wherein said radiation source comprises heavy ion particles.

42. The method of claim 38, wherein said radiation source comprises protons.

43. The method of claim 38, wherein said heavy ion particles comprise iron.

44. The method of claim 38, wherein said radiation source comprises beta particles.

45. The method of claim 38, wherein said radiation source comprises gamma rays.

46. The method of claim 38, wherein said radiation source comprises X-rays.

47. The method of claim 38, wherein said radiation source comprises a radioactive element.

48. The method of claim 47, wherein said radioactive element comprises cobalt.

49. The method of claim 47, wherein said radioactive element comprises uranium.

50. The method of claim 47, wherein said radioactive element comprises plutonium.

51. The method of claim 47, wherein said radioactive element comprises thorium.

52. A method, comprising:
a) providing;
i) a subject exhibiting at least one symptom of a colorectal disease;
ii) a composition comprising a mixture of fish oil and pectin;
b) administering the composition to the subject under conditions such that at least one symptom is reduced.

53. The method of claim 52, wherein said colorectal disease or disorder is selected from the group including, but not limited to, colorectal cancer, colon cancer, large bowel cancer, colonic polyps, anal cancer, general anal and rectal diseases, colitis, Crohn's disease, hemorrhoids, ischemic colitis, ulcerative colitis, diverticulosis, diverticulitis and irritable bowel syndrome.

54. The method of claim 52, wherein said subject is a human.

55. The method of claim 52, wherein said administering comprises dietary consumption.

56. The method of claim 52, wherein said disease development reduction results from increased colorectal cell apoptosis.

57. The method of claim 52, wherein said disease development reduction results from decreased colorectal cell proliferation.

58. The method of claim 56, wherein said increased apoptosis comprises an upregulation of genes associated with the Wnt/β-catenin pathways.

59. The method of claim 57, wherein said decreased cell proliferation comprises an upregulation of genes associated with the Wnt/β-catenin pathways.

60. The method of claim 56, wherein said increased apoptosis comprises a downregulation of tyrosine kinase related genes.

61. The method of claim 57, wherein said decreased cell proliferation comprises a downregulation of tyrosine kinase related genes.

62. The method of claim 61, wherein said tyrosine kinase related genes regulate Ras-PI3K/Akt pathways.

63. The method of claim 52, wherein said subject at risk has been exposed to a carcinogen.

64. The method of claim 52, wherein said subject at risk has been exposed to a radiation source.

65. The method of claim 64, wherein said radiation source comprises cosmic radiation.

66. The method of claim 64, wherein said radiation source comprises a medical device.

67. The method of claim 64, wherein said radiation source comprises heavy ion particles.

68. The method of claim 64, wherein said radiation source comprises protons.

69. The method of claim 64, wherein said heavy ion particles comprise iron.

70. The method of claim 64, wherein said radiation source comprises beta particles.

71. The method of claim 64, wherein said radiation source comprises gamma rays.

72. The method of claim 64, wherein said radiation source comprises X-rays.

73. The method of claim 64, wherein said radiation source comprises a radioactive element.

74. The method of claim 64, wherein said radioactive element comprises cobalt.

75. The method of claim 64, wherein said radioactive element comprises uranium.

76. The method of claim 64, wherein said radioactive element comprises plutonium.

77. The method of claim 64, wherein said radioactive element comprises thorium.

78. A composition comprising a mixture of fish oil and pectin.

79. The composition of claim 78, wherein said composition comprises a tablet.

80. The composition of claim 78, wherein said composition comprises a capsule.

81. The composition of claim 78, wherein said composition comprises a gel.

82. The composition of claim 78, wherein said composition further comprises a meat binder.

83. The composition of claim 78, wherein said composition further comprises a vegetable binder.

84. The composition of claim 78, wherein said composition further comprises a fruit binder.

85. The composition of claim 78, wherein said composition comprises a dairy binder.
